(19) United States
(12) Patent Application Publication
(10) Pub. No.: US 2022/0273593 A1
(43) Pub. Date: Sep. 1, 2022

(54) COMPOUNDS AND COMPOSITIONS FOR TREATING KIDNEY DISEASE

(71) Applicants: JOHANN WOLFGANG GOETHE-UNIVERSITÄT FRANKFURT AM MAIN, FRANKFURT AM MAIN (DE); THE MEDICAL COLLEGE OF WISCONSIN, INC., MILWAUKE, WI (US)

(72) Inventors: JOHN D. IMIG, PEWAUKEE, WI (US); DANIEL MERK, FRANKFURT AM MAIN (DE); ABDUL HYE KHAN, MILWAUKEE, WI (US); MORITZ HELMSTÄDTER, FRANKFURT AM MAIN (DE)

(21) Appl. No.: 17/636,276

(22) PCT Filed: Sep. 21, 2020
(86) PCT No.: PCT/EP2020/076312

§ 371 Date: Feb. 17, 2022

Related U.S. Application Data
(60) Provisional application No. 62/902,771, filed on Sep. 19, 2019.

(51) Int. Cl. A61K 31/18; A61K 31/166; A61P 1/16; A61P 13/12; A61P 17/02

(52) U.S. Cl. CPC A61K 31/18; A61K 31/166; A61P 1/16; A61P 13/12; A61P 17/02

(57) ABSTRACT

The invention is based on a class of dual modulators of soluble epoxide hydrolase (sEH) and farnesoid X receptor (FXR), in particular of compounds having an activity as FXR agonist and sEH inhibitor for the treatment or prevention of kidney diseases and/or fibrotic diseases. The invention provides the compounds for use in such treatments and preventions as well as pharmaceutical compositions comprising the compounds as active ingredients.

### FIELD OF THE INVENTION

The invention is based on a class of dual modulators of soluble epoxide hydrolase (sEH) and farnesoid X receptor (FXR), in particular of compounds having an activity as FXR agonist and sEH inhibitor for the treatment or prevention of kidney diseases and/or fibrotic diseases. The invention provides the compounds for use in such treatments and preventions as well as pharmaceutical compositions comprising the compounds as active ingredients.

### DESCRIPTION

Renal fibrosis causes end-stage renal disease (ESRD), which is the common clinical end point for all progressive renal diseases. The common etiologies of chronic kidney disease and the consequent ESRD include untreated diabetes, hypertension, glomerulonephritis, and chronic pyelonephritis. ESRD is a major burden to the health care system, and most patients are inevitably placed on life-long dialysis and ultimately require transplantation (Liu, 2006; Collins et al., 2009). The ESRD burden on health care is largely due to the lack of an anti-fibrotic agent that can target kidney disease.

Indeed, little success has been made over the past decade in developing antifibrotic therapies to slow the progression of CKD to ESRD (Zhong et al., 2017). Currently, angiotensin-converting enzyme inhibitors and angiotensin II receptor blockers are used to reduce proteinuria and slow CKD progression (Fried et al., 2013). However, these drugs have incomplete efficacy, and trials targeting other mechanisms have failed, including the nuclear factor E2-related factor 2 inducer bardoxolone (De Zeeuw et al., 2013) and the endothelin receptor blocker avosentan (Mann et al., 2010).

WO 2018/215610 A1 (incorporated herein by reference in its entirety) discloses a new class of dual FXR agonist and inhibitor of soluble epoxide hydrolase (sEHi). The disclosure describes methods for treating subjects suffering from diseases associated with FXR using the disclosed new class of compounds. Diseases treatable with the compounds include metabolic disorders, in particular non-alcoholic fatty liver or non-alcoholic steatohepatitis (NASH).

Thus, it is an objective of the invention to provide new treatment options for kidney diseases and/or fibrotic diseases, preferably those which are associated with renal fibrosis.

### BRIEF DESCRIPTION OF THE INVENTION

Generally, and by way of brief description, the main aspects of the present invention can be described as follows:

In a first aspect, the invention pertains to a compound for use in the treatment or prevention of a kidney disease and/or fibrotic disease in a subject, the compound having the formula I:

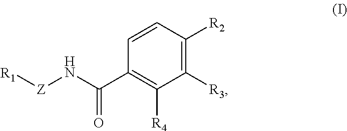

wherein R₁, R₂, R₃ and R₄ are independently selected from H, an unsubstituted, monosubstituted, or polysubstituted C₁-C₁₈ alkyl or heteroalkyl, wherein said alkyl is straight, branched or cyclic, a unsubstituted, monosubstituted or polysubstituted C₁-C₁₈ alkenyl or heteroalkenyl, wherein said alkenyl is straight, branched or cyclic, an unsubstituted, monosubstituted, or polysubstituted aryl or heteroaryl, an unsubstituted, monosubstituted, or polysubstituted benzyl group, an acyl group, such as formyl, acetyl, trichloroacetyl, fumaryl, maleyl, succinyl, benzoyl, or acyl groups being branched, heteroatom-substituted or aryl-substituted, a sugar or another acetal, and a sulfonyl group, and/or R₂, R₃ and/or R₄ form together a nonsubstituted, monosubstituted, or polysubstituted ring, preferably an aromatic ring;
Z is C with or without any substitution;
or an isomer, prodrug, or derivative thereof, or a pharmaceutically acceptable salt or solvate of these compounds.

In a second aspect, the invention pertains to pharmaceutical composition comprising a compound recited in the first aspect, together with a pharmaceutical acceptable carrier and/or excipient.

In a third aspect, the invention pertains to a pharmaceutical composition for use in the treatment or prevention of a kidney disease and/or fibrotic disease, wherein the pharmaceutical composition is as recited in the second aspect.

In a fourth aspect, the invention pertains to a method of treating or preventing a kidney disease and/or a fibrotic disease in subject, the method comprising a step of administering to the subject a therapeutically effective amount of the compound recited in the first aspect, or of the pharmaceutical composition recited in the second aspect.

### DETAILED DESCRIPTION OF THE INVENTION

In the following, the elements of the invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine two or more of the explicitly described embodiments or which combine the one or more of the explicitly described embodiments with any number of the disclosed and/or preferred elements. Furthermore, any permutations and combinations of all described elements in this application should be considered disclosed by the description of the present application unless the context indicates otherwise.

In the first aspect, the invention pertains to

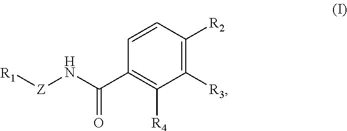

wherein R₁, R₂, R₃ and R₄ are independently selected from H, an unsubstituted, monosubstituted, or polysubstituted C₁-C₁₈ alkyl or heteroalkyl, wherein said alkyl is straight, branched or cyclic, a unsubstituted, monosubstituted or polysubstituted C₁-C₁₈ alkenyl or heteroalkenyl, wherein said alkenyl is straight, branched or cyclic, an unsubstituted, monosubstituted, or polysubstituted aryl or heteroaryl, an unsubstituted, monosubstituted, or polysubstituted benzyl group, an acyl group, such as formyl, acetyl, trichloroacetyl, fumaryl, maleyl, succinyl, benzoyl, or acyl groups being branched, heteroatom-substituted or aryl-substituted, a sugar or another acetal, and a sulfonyl group, and/or wherein R₂, R₃ and/or R₄ form together a nonsubstituted, monosubstituted, or polysubstituted ring, preferably an aromatic ring,
Z is C with or without any substitution, preferably substituted with H or alkyl;
or an isomer, racemate, prodrug, or derivative thereof, or a pharmaceutically acceptable salt or solvate of these compounds.

The compound according to the first aspect is preferably a compound for use in the treatment or prevention of a kidney disease and/or fibrotic disease in a subject.

The compound according to the first aspect is in other embodiments preferably a compound for use in the treatment or prevention of a fibrotic disease in a subject.

In some preferred embodiments R₂ is C₁-C₁₀ alkyl, preferably a branched alky, more preferably the group —C(CH₃)₃, preferably R₃ is H, —OH or OMe, and preferably R₄ is H, —OH or —OMe.

The compound is in another preferred embodiment the above defined group of compounds excluding the herein disclosed compounds 4a, 4b, 6, 7, 9, 14, 16, 19, 29, 33, 36, 42, and 45. In this regard, further preferred is any single one of the other (excluding the above) compounds disclosed in tables 1 to 8 in the example section of this application. In some embodiments the compounds of table 8 are preferred.

In one preferred embodiment the R₃ and R₄ in the above compound is H.

In some embodiments is R₁ a group having the formula II:

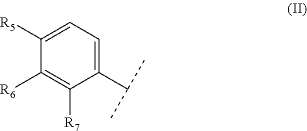

wherein R₅, R₆ and R₇ are independently selected from H, an unsubstituted, monosubstituted, or polysubstituted C₁-C₁₈ alkyl or heteroalkyl, wherein said alkyl is straight, branched or cyclic, a unsubstituted, monosubstituted or polysubstituted C₁-C₁₈ alkenyl or heteroalkenyl, wherein said alkenyl is straight, branched or cyclic, an unsubstituted, monosubstituted, or polysubstituted aryl or heteroaryl, an unsubstituted, monosubstituted, or polysubstituted benzyl group, an acyl group, such as formyl, acetyl, trichloroacetyl, fumaryl, maleyl, succinyl, benzoyl, or acyl groups being branched, heteroatom-substituted or aryl-substituted, a sugar or another acetal, and an unsubstituted, monosubstituted, or polysubstituted amide or sulfonyl group.

Preferably R₆ and R₇ are H or halogen, preferably a halogen selected from F or Cl. Most preferably R₆ is H, and R₇ is H, F or Cl.

Preferably R₅ is a side chain of any length comprising a carboxylic acid or a suitable carboxylic acid replacement such as a typical bioisoster including but not limited to a tetrazole, a sulfonamide, an amide (such as an organic amide, a sulfonamide, or a phosphoramide) or the like.

The term “bioisoster” as used herein relates to a chemical moiety, which replaces another moiety in a molecule of an active compound without significant influence on its biological activity. Other properties of the active compound, such as for example its stability as a medicament, can be affected in this way.

As bioisoster moieties for carboxy (COOH) group can be mentioned especially 5-membered heterocyclic groups having from 1 to 4 heteroatoms selected from nitrogen, oxygen and sulphur, such as for example 1,3,4-oxadiazolyl, 1,2,3-oxadiazolyl, 1,2,5-oxadiazolyl, 1,2,4-oxadiazolyl, 1,3,4-thiadiazolyl, 1,2,4-thiadiazolyl, 1,2,3-thiadiazolyl, 1,2,5-thiadiazolyl, furyl, thienyl, pyrrolyl, pyrazolyl, imidazolyl, isoxazolyl, isothiazolyl, and N-substituted tetrazolyl. 5-Membered heterocyclic groups can be optionally substituted with 1 or 2 substituents selected from the group comprising phenyl, pyridinyl, straight or branched alkyl group, amino group, hydroxy group, fluoro, chloro, bromo, iodo, trifluoromethyl, trifluoromethoxy, trifluorothiomethoxy, alkoxy, and thioalkoxy.

As bioisoster moieties for carboxy (COOH) group can be also mentioned phenyl and 6-membered heterocyclic groups having from 1 to 4 heteroatoms selected from nitrogen, oxygen and sulphur, such as for example pyridinyl, pyrazinyl, pyridazinyl, pyrimidinyl, triazinyl, tetrazinyl, and others. Phenyl and 6-membered heterocyclic groups can be optionally substituted with 1 or 2 substituents selected from the group comprising phenyl, pyridinyl, straight or branched alkyl group, amino group, hydroxy group, fluoro, chloro, bromo, iodo, trifluoromethyl, trifluoromethoxy, trifluorothiomethoxy, alkoxy, and thioalkoxy.

In some embodiments of the invention R₁ is selected from any of the following substituents:

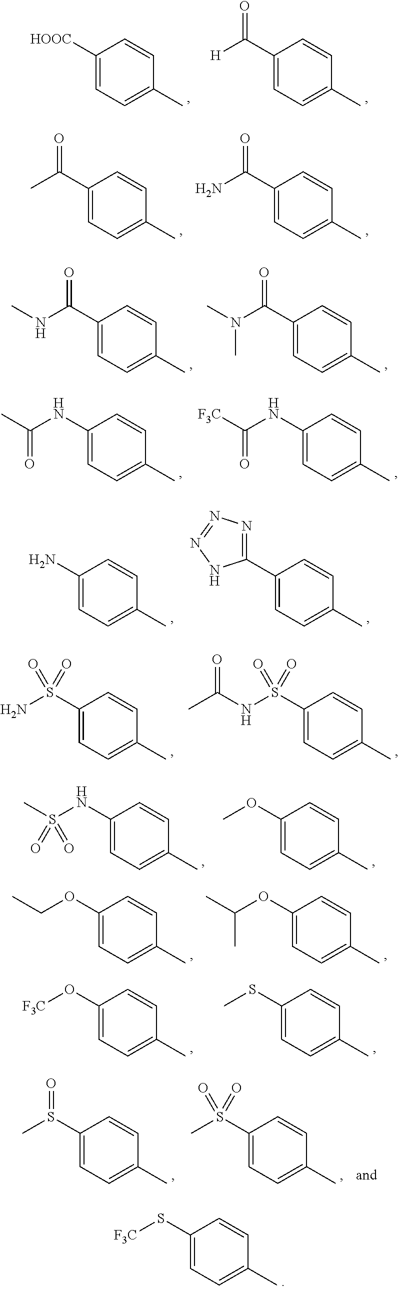

Most preferably the compound of the invention has the above formula I, wherein R₁ is selected from the group consisting of:

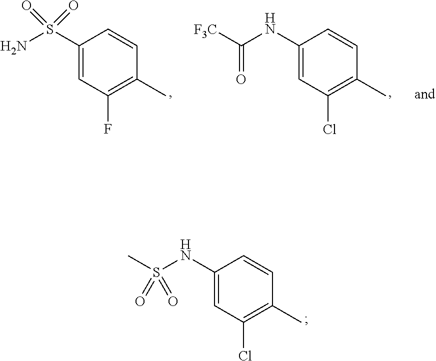

and
wherein Z is C, R₂ is —C(CH₃)₃, and R₄, R₃ is H.

In another embodiment, further preferred is the above compound having formula I and wherein R₁ is selected from the group consisting of:

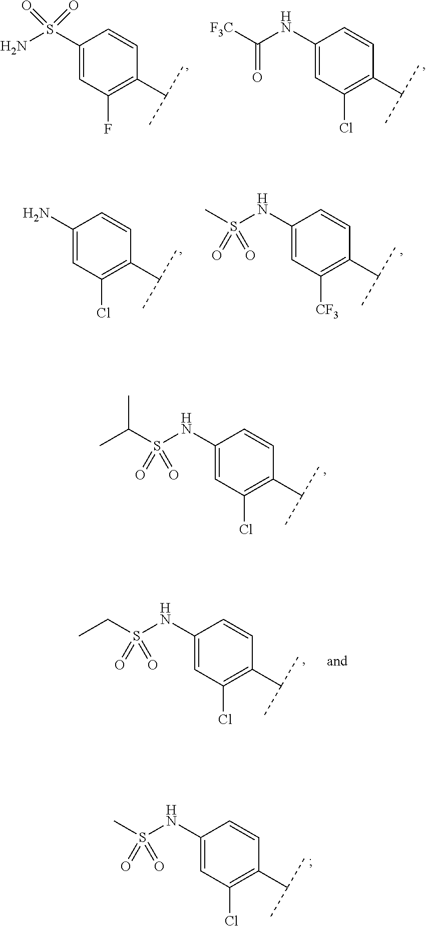

and
wherein Z is C, R₃ is H or OH, and R₄ is H or OH, in particular R₃ and R₄ are not both OH; and
wherein R₂ is selected from —C(CH₃)₃, —N(CH₃)₂, or the R₂ is any of the following

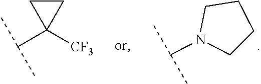

The compound according to the present disclosure in the most preferred embodiments is a compound with the following formula:

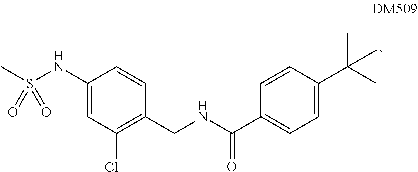 [SMILES: CC(C)(C)c1ccc(C(=O)NCc2ccc(NS(C)(=O)=O)cc2Cl)cc1]

or an isomer, prodrug, or derivative thereof, or a pharmaceutically acceptable salt or solvate of this compound. The compound as well as an isomer, prodrug, or derivative thereof, or a pharmaceutically acceptable salt or solvate of this compound is herein also referred to as DM509.

In an alternative first aspect, the invention pertains to an alternative compound having the following formula (Ib)

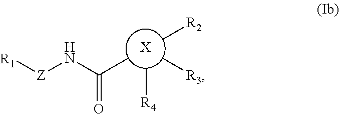

wherein R₁, R₂, R₃ and R₄ are independently selected from H, an unsubstituted, monosubstituted, or polysubstituted C₁-C₁₈ alkyl or heteroalkyl, wherein said alkyl is straight, branched or cyclic, a unsubstituted, monosubstituted or polysubstituted C₁-C₁₈ alkenyl or heteroalkenyl, wherein said alkenyl is straight, branched or cyclic, an unsubstituted, monosubstituted, or polysubstituted aryl or heteroaryl, an unsubstituted, monosubstituted, or polysubstituted benzyl group, an acyl group, such as formyl, acetyl, trichloroacetyl, fumaryl, maleyl, succinyl, benzoyl, or acyl groups being branched, heteroatom-substituted or aryl-substituted, a sugar or another acetal, and a sulfonyl group, and/or wherein R₂, R₃ and/or R₄ form together a nonsubstituted, monosubstituted, or polysubstituted ring, preferably an aromatic ring,

X is aryl, preferably selected from selected from the group consisting of thiophene, thiazole, isothiazole, 1,2,3-thiadiazole, 1,2,4-thiadiazole, 1,2,5-thiadiazole, 1,3,4-thiadiazole, pyrrole, imidazole, pyrazole, 1H-1,2,3-triazole, 2H-1,2,3-triazole, 1H-1,2,4-triazole, furan, oxazole, isoxazole, 1,2,3-oxadiazole, 1,2,4-oxadiazole, 1,2,5-oxadiazole, 1,3,4-oxadiazole, pyridine, pyridazine, pyrimidine, and pyrazine; or X is selected from any one of the following structures:

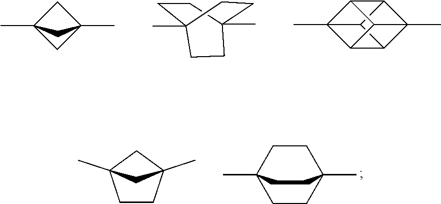

Z is C with or without any substitution, preferably substituted with H or alkyl;
or an isomer, racemate, prodrug, or derivative thereof, or a pharmaceutically acceptable salt or solvate of these compounds.

The compound according to the first aspect is preferably a compound for use in the treatment or prevention of a kidney disease and/or fibrotic disease in a subject.

Preferably X is a thiophene.

The compound according to the first aspect is in other embodiments preferably a compound for use in the treatment or prevention of a fibrotic disease in a subject.

In some preferred embodiments R₂ is C₁-C₁₀ alkyl, preferably a branched alky, more preferably the group —C(CH₃)₃, preferably R₃ is H, —OH or OMe, and preferably R₄ is H, —OH or —OMe.

The compound is in another preferred embodiment the above defined group of compounds excluding the herein disclosed compounds 4a, 4b, 6, 7, 9, 14, 16, 19, 29, 33, 36, 42, and 45. In this regard, further preferred is any single one of the other (excluding the above) compounds disclosed in tables 1 to 8 in the example section of this application. In some embodiments the compounds of table 8 are preferred.

In one preferred embodiment the R₃ and R₄ in the above compound is H.

In some embodiments is R₁ a group having the formula II:

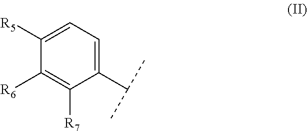

wherein R₅, R₆ and R₇ are independently selected from H, an unsubstituted, monosubstituted, or polysubstituted C₁-C₁₈ alkyl or heteroalkyl, wherein said alkyl is straight, branched or cyclic, a unsubstituted, monosubstituted or polysubstituted C₁-C₁₈ alkenyl or heteroalkenyl, wherein said alkenyl is straight, branched or cyclic, an unsubstituted, monosubstituted, or polysubstituted aryl or heteroaryl, an unsubstituted, monosubstituted, or polysubstituted benzyl group, an acyl group, such as formyl, acetyl, trichloroacetyl, fumaryl, maleyl, succinyl, benzoyl, or acyl groups being branched, heteroatom-substituted or aryl-substituted, a sugar or another acetal, and an unsubstituted, monosubstituted, or polysubstituted amide or sulfonyl group.

Preferably R₆ and R₇ are H or halogen, preferably a halogen selected from F or Cl. Most preferably R₆ is H, and R₇ is H, F or Cl.

Preferably R₅ is a side chain of any length comprising a carboxylic acid or a suitable carboxylic acid replacement such as a typical bioisoster including but not limited to a tetrazole, a sulfonamide, an amide (such as an organic amide, a sulfonamide, or a phosphoramide) or the like.

The term “bioisoster” as used herein relates to a chemical moiety, which replaces another moiety in a molecule of an active compound without significant influence on its biological activity. Other properties of the active compound, such as for example its stability as a medicament, can be affected in this way.

As bioisoster moieties for carboxy (COOH) group can be mentioned especially 5-membered heterocyclic groups having from 1 to 4 heteroatoms selected from nitrogen, oxygen and sulphur, such as for example 1,3,4-oxadiazolyl, 1,2,3-oxadiazolyl, 1,2,5-oxadiazolyl, 1,2,4-oxadiazolyl, 1,3,4-thiadiazolyl, 1,2,4-thiadiazolyl, 1,2,3-thiadiazolyl, 1,2,5-thiadiazolyl, furyl, thienyl, pyrrolyl, pyrazolyl, imidazolyl, isoxazolyl, isothiazolyl, and N-substituted tetrazolyl. 5-Membered heterocyclic groups can be optionally substituted with 1 or 2 substituents selected from the group comprising phenyl, pyridinyl, straight or branched alkyl group, amino group, hydroxy group, fluoro, chloro, bromo, iodo, trifluoromethyl, trifluoromethoxy, trifluorothiomethoxy, alkoxy, and thioalkoxy.

As bioisoster moieties for carboxy (COOH) group can be also mentioned phenyl and 6-membered heterocyclic groups having from 1 to 4 heteroatoms selected from nitrogen, oxygen and sulphur, such as for example pyridinyl, pyrazinyl, pyridazinyl, pyrimidinyl, triazinyl, tetrazinyl, and others. Phenyl and 6-membered heterocyclic groups can be optionally substituted with 1 or 2 substituents selected from the group comprising phenyl, pyridinyl, straight or branched alkyl group, amino group, hydroxy group, fluoro, chloro, bromo, iodo, trifluoromethyl, trifluoromethoxy, trifluorothiomethoxy, alkoxy, and thioalkoxy.

In some embodiments of the invention R₁ is selected from any of the following substituents:

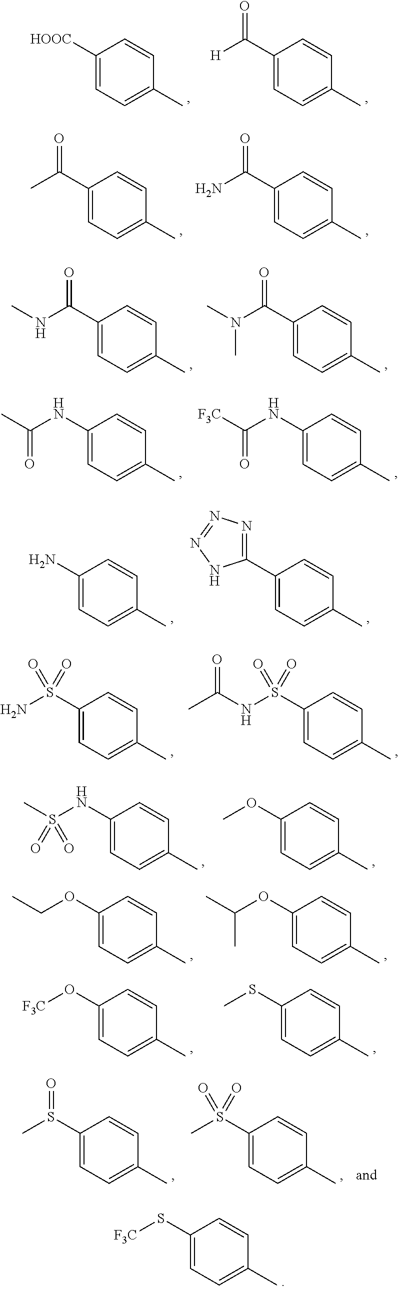

Most preferably the compound of the invention has the above formula I, wherein R₁ is selected from the group consisting of:

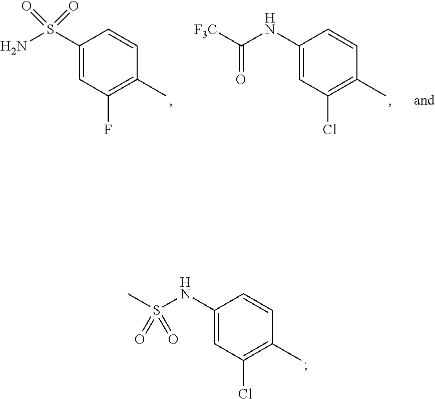

and
wherein Z is C, R₂ is —C(CH₃)₃, and R₄, R₃ is H.

In another embodiment, further preferred is the above compound having formula I and wherein R₁ is selected from the group consisting of:

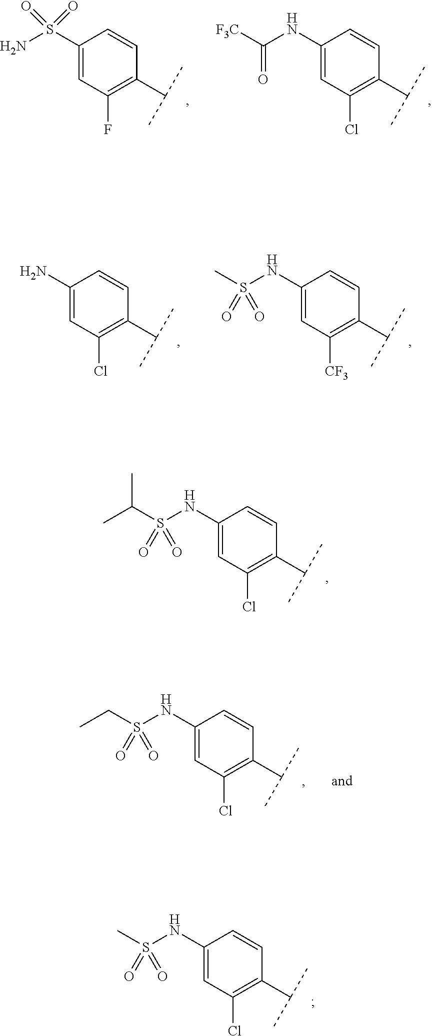

and
wherein Z is C, R₃ is H or OH, and R₄ is H or OH, in particular R₃ and R₄ are not both OH; and
wherein R₂ is selected from —C(CH₃)₃, —N(CH₃)₂, or the R₂ is any of the following

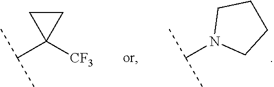

The compound according to the present disclosure in the most preferred embodiments is a compound with the following formula:

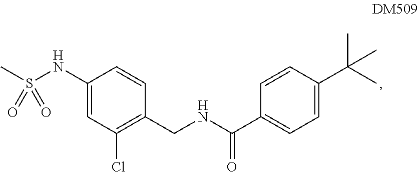 [SMILES: CC(C)(C)c1ccc(C(=O)NCc2ccc(NS(C)(=O)=O)cc2Cl)cc1]

or an isomer, prodrug, or derivative thereof, or a pharmaceutically acceptable salt or solvate of this compound. The compound as well as an isomer, prodrug, or derivative thereof, or a pharmaceutically acceptable salt or solvate of this compound is herein also referred to as DM509 (or more simply “DM”).

In another preferred embodiment of the alternative aspect the present invention pertains to a compound having the following formular (III):

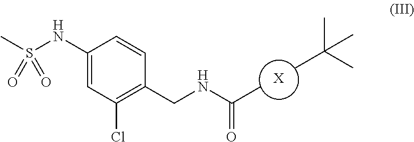

wherein X is as defined herein above,
or an isomer, prodrug, or derivative thereof, or a pharmaceutically acceptable salt or solvate of this compound. The compound as well as an isomer, prodrug, or derivative thereof.

The compound according to the present disclosure in another alternative most preferred embodiments is a compound with the following formula:

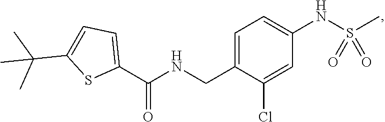 [SMILES: CC(C)(C)c1ccc(C(=O)NCc2ccc(NS(C)(=O)=O)cc2Cl)s1]

or an isomer, prodrug, or derivative thereof, or a pharmaceutically acceptable salt or solvate of this compound. The compound as well as an isomer, prodrug, or derivative thereof, or a pharmaceutically acceptable salt or solvate of this compound is herein also referred to as HM.

The following descriptions pertain to both the first and the alternative first aspect of the invention:

The compound according to the invention is preferably a (dual) farnesoid X receptor (FXR) agonist and a soluble epoxide hydrolase (sEH) inhibitor.

Salts with a pharmaceutically unacceptable anion likewise form part of the scope of the invention as useful intermediates for the preparation or purification of pharmaceutically acceptable salts and/or for use in nontherapeutic, for example in vitro, applications. The compounds of the invention may also exist in various polymorphous forms, for example as amorphous and crystalline polymorphous forms. All polymorphous forms of the inventive compounds are within the scope of the invention and are a further aspect of the invention.

As used herein, the term “farnesoid X receptor” or “FXR” refers to all mammalian forms of such receptor including, for example, alternative splice isoforms and naturally occurring isoforms (see e.g. R. M. Huber et al., Gene 2002, 290, 35). Representative FXR species include, without limitation the rat (GenBank Accession No. NM_21745), mouse (Genbank Accession No. NM_09108), and human (GenBank Accession No. NM_05123) forms of the receptor.

The term “soluble epoxide hydrolase (sEH)” is meant to refer to a bifunctional enzyme that in humans is encoded by the EPHX2 gene (HGNC:3402). sEH is a member of the epoxide hydrolase family. This enzyme, found in both the cytosol and peroxisomes, binds to specific epoxides and converts them to the corresponding diols.

Another aspect then relates to a method of treating a disease in subject, the method comprising a step of administering to the subject a therapeutically effective amount of the compound of the invention, or of the pharmaceutical composition of the invention.

In context of the present disclosure the term “subject” preferably pertains to a mammal, preferably a mouse, rat, donkey, horse, cat, dog, guinea pig, monkey, ape, or preferably to a human patient, for example a patient suffering from the herein described disorders.

The compounds of the invention are in particular useful in a method of treating a kidney disease and/or fibrotic disease in a subject. Preferably the kidney disease and/or fibrotic disease to be treated according to the invention is a disorder associated with FXR and sEH.

The term “kidney disease” as used herein refers to disorders associated with any stage or degree of acute or chronic renal failure that results in a loss of the kidney's ability to perform the function of blood filtration and elimination of excess fluid, electrolytes, and wastes from the blood. Kidney disease also includes endocrine dysfunctions such as anemia (erythropoietin-deficiency), and mineral imbalance (Vitamin D deficiency). Kidney disease may originate in the kidney or may be secondary to a variety of conditions, including (but not limited to) heart failure, hypertension, diabetes, autoimmune disease, or liver disease. Kidney disease may be a condition of chronic renal failure that develops after an acute injury to the kidney. For example, injury to the kidney by ischemia and/or exposure to toxicants may cause acute renal failure; incomplete recovery after acute kidney injury may lead to the development of chronic renal failure.

The term “treatment” refers to both therapeutic treatment and prophylactic or preventative measures for kidney disease and/or fibrotic disease, anemia, tubular transport deficiency, or glomerular filtration deficiency wherein the object is to reverse, prevent or slow down (lessen) the targeted disorder. Those in need of treatment include those already having a kidney disease, anemia, tubular transport deficiency, or glomerular filtration deficiency as well as those prone to having a kidney disease, anemia, tubular transport deficiency, or glomerular filtration deficiency or those in whom the kidney disease, anemia, tubular transport deficiency, or glomerular filtration deficiency is to be prevented. The term “treatment” as used herein includes the stabilization and/or improvement of kidney function.

The term “chronic kidney disease” or “CKD” is used herein interchangeably to refer to a condition defined as abnormalities of kidney structure or function, present for more than 3 months, with implications for health which can occur abruptly, and either resolve or become chronic (Clinical Practice Guideline for the Evaluation and Management of Chronic Kidney Disease Guidelines (KDIGO 2012). CKD is a general term for heterogeneous disorders affecting kidney structure and function with variable clinical presentation, in part related to cause, severity and the rate of progression (Kidney International Supplements (2013) 3, vii). In preferred embodiments of the invention the kidney disease is a chronic kidney disease.

Also provided is, thus, a method for concomitant modulation of FXR and sEH, the method comprising the step of administering to a subject or a cell a dual FXR and sEH modulator as described herein before.

In yet another embodiment the kidney disease is a progression of CKD, preferably a progression to end stage renal disease (ESRD). The term “progression of CKD” in context of the present invention is understood to refer to a worsening of any of the pathological hallmarks of CKD in a patient. The present invention preferably relates to a use of the compounds as disclosed to alleviate such progression and thus a treatment of the invention may in some embodiments be understood as a prevention of progression of CKD, or preferably as a prevention or treatment of ESRD.

As used herein, the term “end-stage renal disease” is to be understood as referring to a condition wherein the subject has permanently lost substantial renal function, for example, having a glomerular filtration rate below 15 ml/min/1.73 m², or the subject is receiving indefinite renal replacement therapy (eg dialysis such as haemodialysis or peritoneal dialysis).

In further particular embodiments the kidney disease treatable or preventable according to the aspects and embodiments of the invention is, or involves in its pathology and one or any combination of the following conditions: renal fibrosis, renal inflammation, and/or renal tubular injury.

Generally the term “renal fibrosis” refers to a situation where any kind or fibrotic condition in the kidney of a subject has arisen. Renal fibrosis usually involves excessive proliferation of cells, hardening tissue and scarring of kidney tissue.

As herein defined, the term “renal inflammation” extends to all conditions which are substantially characterised by the occurrence of inflammation within the kidney, or where the occurrence of inflammation in the kidney is caused by a disease or an inflammatory condition which primarily affects a site in the body other than the kidney. In particular, inflammation may occur at a site including, but not limited to; the glomerulus, Bowman's capsule or Bowman's space. Typically, the inflammation results in at least partial impairment of kidney function and/or kidney failure. Renal inflammation is preferably characterized by any one or a combination of: increased TNF-alpha, increased IL1β, increased IL6, increased fibronectin expression, and increased alpha-SMA in the kidney.

In further particular embodiments, the treatments and preventions of the invention are characterized in that they reduce and/or mitigate renal epithelial to mesenchymal transition.

As used herein, the term “epithelial to mesenchymal transition” (EMT) refers to the conversion of a cell from an epithelial to a mesenchymal phenotype, which is a normal process of embryonic development. EMT is also the process whereby injured epithelial cells that function as ion and fluid transporters become matrix remodeling mesenchymal cells. The criteria for defining EMT in vitro involve the loss of epithelial cell polarity, the separation into individual cells and subsequent dispersion after the acquisition of cell motility (see Vincent-Salomon et al., Breast Cancer Res. 2003; 5(2): 101-106). Growth factors including, but not limited to, TGF-β (e.g., TGF-β1, TGF-β2, TGF-β3 and Wnts (e.g., Wnt1, Wnt3A, Wnt8, Wnt10a) and transcription factors including, but not limited to, LEF and β-catenin are causally involved in regulating EMT (see Thompson et al., Cancer Research 65, 5991-5995, Jul. 15, 2005).

As used herein, the term “endothelial to mesenchymal transition” (EnMT) refers to the phenotypic conversion of endothelial cells to a mesenchymal-myofibroblast phenotype.

As used herein, the term “epithelium” refers to the covering of internal and external surfaces of the body, including the lining of vessels and other small cavities. It consists of a collection of epithelial cells forming a relatively thin sheet or layer due to the constituent cells being mutually and extensively adherent laterally by cell-to-cell junctions. The layer is polarized and has apical and basal sides. Despite the tight regimentation of the epithelial cells the epithelium does have some plasticity and cells in an epithelial layer can alter shape, such as change from flat to columnar, or pinch in at one end and expand at the other. However, these tend to occur in cell groups rather than individually (see Thompson et al., Cancer Research 65, 5991-5995, Jul. 15, 2005).

As used herein, the term “mesenchyme” refers to the part of the embryonic mesoderm, consisting of loosely packed, unspecialized cells set in a gelatinous ground substance, from which connective tissue, bone, cartilage, and the circulatory and lymphatic systems develop. Mesenchyme is a collection of cells which form a relatively diffuse tissue network. Mesenchyme is not a complete cellular layer and the cells typically have only points on their surface engaged in adhesion to their neighbors. These adhesions may also involve cadherin associations (see Thompson et al., Cancer Research 65, 5991-5995, Jul. 15, 2005).

As used herein, the term “myofibroblast” refers to fibroblasts that are associated with the increased and often pathological deposition of ECM at fibrotic lesions. Myofibroblasts are activated in response to injury or increased epithelial to mesenchymal crosstalk and are thought to be the primary producers of ECM components following injury. Myofibroblasts originate from differentiation of resident mesenchymal fibroblasts (hepatic stellate cells in the liver), from EMT, and from EnMT. Myofibroblast differentiation is an early event in the development of fibrosis. Myofibroblast-like cells express smooth muscle (SM) cytoskeletal markers (α-SM actin in particular) and participate actively in the production of extracellular matrix.

Increased EMT/EnMT, prolonged myofibroblast activation, and increased deposition of extracellular matrix are features common to many fibroproliferative diseases, including but not limited to pulmonary fibrosis, liver fibrosis, kidney fibrosis, systemic sclerosis, and fibrosis arising from transplant rejection. The spectrum of affected organs, the usually progressive nature of the fibrotic process, the large number of affected persons, and the absence of effective treatment pose an enormous challenge when treating fibrotic diseases. Current treatments for fibrotic diseases typically target the inflammatory response, but there is accumulating evidence that the mechanisms driving fibrosis are distinct from those regulating inflammation. In fact, some studies suggest that ongoing inflammation reverses established and progressive fibrosis.

A fibrotic disease treatable or preventable according to the present invention is a condition where the fibroproliferative response produces an abnormal accumulation of fibrocellular scar tissue that compromises the normal architecture and function of the affected tissue. Non-limiting examples of fibrotic disease include Peyronie's disease, Raynaud's syndrome, psoriasis plaques, eczema, keloid scars, pulmonary fibrosis, liver fibrosis, renal fibrosis, and vascular fibrosis. Renal fibrosis is preferred however. The fibrotic disease is preferably characterized by increased EMT/EnMT as described above.

In some embodiments which are preferred, and in particular in context with the alternative aspect of the invention, the compounds disclosed herein, and in particular the alternative compounds, are for use in the treatment of a liver disease.

As used herein, the term “liver disease” refers to any disease or disorder that affects the liver. Examples of liver disease include, but are not limited to, Alagille Syndrome; Alcohol-Related Liver Disease; Alpha-1 Antitrypsin Deficiency; Autoimmune Hepatitis; Benign Liver Tumors; Biliary Atresia; Cirrhosis; Galactosemia; Gilbert Syndrome; Hemochromatosis; Hepatitis A; Hepatitis B; Hepatitis C; Hepatocellular Carcinoma; Hepatic Encephalopathy; Liver Cysts; Liver Cancer; Newborn Jaundice; Non- Alcoholic Fatty Liver Disease (including nonalcoholic fatty liver and nonalcoholic steatohepatitis); Primary Biliary Cirrhosis (PBC); Primary Sclerosing Cholangitis (PSC); Reye Syndrome; Type I Glycogen Storage Disease and Wilson Disease.

The terms “Non-alcoholic fatty liver” or “Non-alcoholic fatty liver disease” (NAFLD) refers to a condition which is one cause of a fatty liver, occurring when fat is deposited in the liver not due to excessive alcohol use. NAFLD is related to insulin resistance and the metabolic syndrome and may respond to treatments originally developed for other insulin-resistant states (e.g. diabetes mellitus type 2) such as weight loss, metformin and thiazolidinediones. NAFLD can be sub-classified as non-alcoholic steatohepatitis (NASH) and nonalcoholic fatty liver (NAFL). NASH is the more extreme form of NAFLD, and is regarded as a major cause of cirrhosis of the liver of unknown cause.

Most patients with NAFLD have few or no symptoms. Patients may complain of fatigue, malaise, and dull right-upper-quadrant abdominal discomfort. Mild jaundice may be noticed although this is rare. More commonly NAFLD is diagnosed following abnormal liver function tests during routine blood tests. NAFLD is associated with insulin resistance and metabolic syndrome (obe-sity, combined hyperlipidemia, diabetes mellitus (type II) and high blood pressure). Common findings are elevated liver enzymes and a liver ultrasound showing steatosis. An ultrasound may also be used to exclude gallstone problems (cholelithiasis). A liver biopsy (tissue examination) is the only test widely accepted as definitively distinguishing NASH from other forms of liver dis-ease and can be used to assess the severity of the inflammation and resultant fibrosis.

Nonalcoholic steatohepatitis (NASH) is a common, often “silent” liver disease. The major fea-ture in NASH is fat in the liver, along with inflammation and damage. Most people with NASH feel well and are not aware that they have a liver problem. Nevertheless, NASH can be severe and can lead to cirrhosis, in which the liver is permanently damaged and scarred and no longer able to work properly.

NASH is usually first suspected in a person who is found to have elevations in liver tests that are included in routine blood test panels, such as alanine aminotransferase (ALT) or aspartate ami-notransferase (AST). When further evaluation shows no apparent reason for liver disease and when x rays or imaging studies of the liver show fat, NASH is suspected. The only means of providing a definitive diagnosis of NASH and separating it from simple fatty liver is a liver biop-sy. NASH is diagnosed when fat along with inflammation and damage to liver cells is observed from the biopsy. If the tissue shows fat without inflammation and damage, NAFL or NAFLD is diagnosed. Currently, no blood tests or scans can reliably provide this information.

Therefore, preferred diseases to be treated with the compounds of the invention are liver diseas-es, such as non-alcoholic fatty liver disease or non-alcoholic steatohepatitis (NASH).

As used herein, the term “therapeutically effective amount” means that amount of a compound that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder.

In yet another aspect there is provided a pharmaceutical composition comprising a compound of the invention together with a pharmaceutically acceptable carrier and/or excipient. Preferably the composition is for use in the treatment or prevention of a kidney disease and/or fibrotic disease. In such compositions of the invention the compound is present in an amount which is when the pharmaceutical composition is administered to a subject therapeutically effective to treat a kidney disease.

The compound(s) of the invention can also be administered in combination with further active ingredients. The amount of a compound of the formula I required to achieve the desired biological effect depends on a number of factors, for example the specific compound chosen, the intended use, the mode of administration and the clinical condition of the patient.

The daily dose is generally in the range from 0.3 mg to 100 mg (typically from 3 mg to 50 mg) per day per kilogram of body weight, for example 3-10 mg/kg/day. An intravenous dose may be, for example, in the range from 0.3 mg to 1.0 mg/kg, which can suitably be administered as infusion of 10 ng to 100 ng per kilogram of body weight per minute. Suitable infusion solutions for these purposes may contain, for example, 0.1 ng to 100 mg, typically 1 ng to 100 mg, per milliliter. Single doses may contain, for example, 1 mg to 10 g of the active ingredient. Thus, ampoules for injections may contain, for example, from 1 mg to 100 mg, and orally administrable single-dose formulations, for example tablets or capsules, may contain, for example, from 1.0 to 1000 mg, typically from 10 to 600 mg. For treatment of the abovementioned conditions, the compounds of the formula I themselves may be used as the compound, but they are preferably present with a compatible carrier in the form of a pharmaceutical composition. The carrier must of course be acceptable in the sense that it is compatible with the other constituents of the composition and is not harmful to the patient's health. The carrier may be a solid or a liquid or both and is preferably formulated with the compound as a single dose, for example as a tablet, which may contain from 0.05% to 95% by weight of the active ingredient. Other pharmaceutically active substances may likewise be present, including other compounds of formula I. The inventive pharmaceutical compositions can be produced by one of the known pharmaceutical methods, which essentially involve mixing the ingredients with pharmacologically acceptable carriers and/or excipients.

Inventive pharmaceutical compositions are those suitable for oral, rectal, topical, peroral (for example sublingual) and parenteral (for example subcutaneous, intramuscular, intradermal or intravenous) administration, although the most suitable mode of administration depends in each individual case on the nature and severity of the condition to be treated and on the nature of the compound of formula I used in each case. Coated formulations or medicament forms are also within the scope of the invention. Sugar-coated formulations and sugar-coated slow-release formulations are also within the scope of the invention. Preference is given to acid- and gastric juice-resistant formulations. Suitable gastric juice-resistant coatings comprise cellulose acetate phthalate, polyvinyl acetate phthalate, hydroxypropylmethylcellulose phthalate and anionic polymers of methacrylic acid and methyl methacrylate. Suitable pharmaceutical compounds for oral administration may be in the form of separate, i.e. single-dose, units, for example capsules, cachets, lozenges, film tablets, sugar-coated tablets, soluble tablets, sublingual tablets, oral tablets or tablets, each of which contains a defined amount of the compound of formula I; as powders or granules; as solution or suspension in an aqueous or nonaqueous liquid; or as an oil-in-water or water-in-oil emulsion. These compositions may, as already mentioned, be prepared by any suitable pharmaceutical method which includes a step in which the active ingredient and the carrier (which may consist of one or more additional ingredients) are brought into contact. The compositions are generally produced by uniform and homogeneous mixing of the active ingredient with a liquid and/or finely divided solid carrier, after which the product is shaped if necessary. For example, a tablet can be produced by compressing or molding a powder or granules of the compound, where appropriate with one or more additional ingredients. Compressed tablets can be produced by tableting the compound in free-flowing form such as, for example, a powder or granules, where appropriate mixed with a binder, glidant, inert diluent (filler) and/or one (or more) surfactant(s)/dispersant(s) in a suitable machine. Molded tablets or granules can be produced by molding the pulverulent compound moistened with an inert liquid diluent in a suitable machine.

Pharmaceutical compositions suitable for peroral (sublingual) administration include lozenges which contain a compound of formula I with a flavoring, typically sucrose, and gum arabic or tragacanth, and pastilles which comprise the compound in an inert base such as gelatin and glycerol or sucrose and gum arabic.

Pharmaceutical compositions suitable for parenteral administration comprise preferably sterile aqueous preparations of a compound of formula I, which are preferably isotonic with the blood of the intended recipient. These preparations are preferably administered intravenously, although administration may also take place by subcutaneous, intramuscular or intradermal injection. These preparations can preferably be produced by mixing the compound with water and making the resulting solution sterile by a suitable sterilization process (e.g. steam sterilization, sterile filtration) and isotonic with blood. Injectable compositions of the invention generally contain from 0.1 to 5% by weight of the active compound. Pharmaceutical compositions suitable for rectal administration are preferably in the form of single-dose suppositories. These can be produced by mixing a compound of formula I with one or more conventional solid carriers, for example cocoa butter, and shaping the resulting mixture.

Pharmaceutical compositions suitable for topical use on the skin are preferably in the form of ointment, cream, powder, lotion, paste, spray, aerosol or oil. Carriers which can be used are petrolatum, lanolin, polyethylene glycols, alcohols and combinations of two or more of these substances. The active ingredient is generally present in a concentration of 0.1 to 15% by weight of the composition, for example 0.5 to 2%.

Transdermal administration is also possible. Pharmaceutical compositions suitable for transdermal uses may be in the form of single patches which are suitable for long-term close contact with the patient's epidermis. Such patches suitably contain the active ingredient in an aqueous solution which is buffered where appropriate, dissolved and/or dispersed in an adhesive or dispersed in a polymer. A suitable active ingredient concentration is about 1% to 35%, preferably about 3% to 15%. A particular option is for the active ingredient to be released by electrotransport or iontophoresis as described, for example, in Pharmaceutical Research, 2(6): 318 (1986).

In another aspect, the invention pertains to a method of treating or preventing a kidney disease and/or a fibrotic disease in subject, the method comprising a step of administering to the subject a therapeutically effective amount of the compound recited in the first aspect, or of the pharmaceutical composition recited in the second aspect.

The terms “of the [present] invention”, “in accordance with the invention”, “according to the invention” and the like, as used herein are intended to refer to all aspects and embodiments of the invention described and/or claimed herein.

As used herein, the term “comprising” is to be construed as encompassing both “including” and “consisting of”, both meanings being specifically intended, and hence individually disclosed embodiments in accordance with the present invention. Where used herein, “and/or” is to be taken as specific disclosure of each of the two specified features or components with or without the other. For example, “A and/or B” is to be taken as specific disclosure of each of (i) A, (ii) B and (iii) A and B, just as if each is set out individually herein. In the context of the present invention, the terms “about” and “approximately” denote an interval of accuracy that the person skilled in the art will understand to still ensure the technical effect of the feature in question. The term typically indicates deviation from the indicated numerical value by ±20%, ±15%, ±10%, and for example ±5%. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect. Where an indefinite or definite article is used when referring to a singular noun, e.g. “a”, “an” or “the”, this includes a plural of that noun unless something else is specifically stated.

It is to be understood that application of the teachings of the present invention to a specific problem or environment, and the inclusion of variations of the present invention or additional features thereto (such as further aspects and embodiments), will be within the capabilities of one having ordinary skill in the art in light of the teachings contained herein.

Unless context dictates otherwise, the descriptions and definitions of the features set out above are not limited to any particular aspect or embodiment of the invention and apply equally to all aspects and embodiments which are described.

All references, patents, and publications cited herein are hereby incorporated by reference in their entirety.

### BRIEF DESCRIPTION OF THE FIGURES

The figures show:

FIG. 1: shows the experimental protocol and effect of DM509 on collagen positive area.

FIG. 2: shows the effect of DM509 treatment on fibrotic inflammation markers.

FIG. 3: shows the experimental protocol and effect of DM509 on collagen positive kidney area and kidney hydroxyprolin.

FIG. 4: shows the effect of DM509 treatment on EMT.

FIG. 5: shows the effect of DM509 treatment on mRNA expressions of claudins 1,3,4 and 5 in the kidney.

FIG. 6: shows the effect of DM509 treatment in inflammation in the kidney.

FIG. 7: shows the effect of DM509 treatment on vascular injury.

FIG. 8: shows (a) schematic overview over the DIO NASH model. (b) Body weight and food intake for the four treatment groups. (c) Biochemical parameters after four weeks of treatment. (d) Biochemical parameters after twelve weeks of treatment.

FIG. 7: shows representative post-treatment histology images for the different treatment groups in the DIO NASH model.

FIG. 9: shows representative pre-/post-treatment histology images for the different treatment groups in the DIO NASH model. Pre- and post-treatment histology images are from identical animals in each group.

FIG. 10: shows representative pre-/post-treatment histology images for the different treatment groups in the DIO NASH model. Pre- and post-treatment histology images are from identical animals in each group.

FIG. 11: shows changes in clinical NASH scores for the four treatment groups. Lean animals for comparison. One-sided Fisher's exact test with Bonferroni correction, *p<0.05, **p<0.01, ***p<0.001 compared to vehicle treated animals.

FIG. 12: shows (a) individual changes in fibrosis stage. (b) Individual changes in liver PSR. Paired t-test, *p<0.05, **p<0.01.

FIG. 13: shows (a) individual changes in lobular inflammation scores. (b) Liver inflammatory parameters. Two-way ANOVA, *p<0.05.

### EXAMPLES

Certain aspects and embodiments of the invention will now be illustrated by way of example and with reference to the description, figures and tables set out herein. Such examples of the methods, uses and other aspects of the present invention are representative only, and should not be taken to limit the scope of the present invention to only such representative examples.

Synthesis of the compounds of the invention is described in

The examples show:

### Comparative Example 1: Synthesis of Compounds

N-benzyl benzamides 4-57 and 77-78 were prepared according to schemes 3-9. Synthesis of aminomethylbenzene precursors 58a-j started with radical bromination of the respective methylbenzene derivatives 59a-j using NBS and AIBN to bromomethylbenzenes 60a-j. Subsequently, bromomethylbenzenes 64a-j were applied to a two-step Staudinger reaction using sodium azide to generate azides 61a-j and triphenylphosphine in water for their reduction. Aminomethylbenzene derivative 58k was prepared by reduction of 4-amino-2-chlorobenzonitrile 62 using LiAlH₄. Aminomethylbenzene derivatives 58l-w were commercially available. Subsequently, 58a-w were reacted with carbonyl chlorides 63a-o in presence of pyridine or with carboxylic acids 64a-f in presence of EDC and 4-DMAP to yield compounds 18, 19, 22, 35, 36, 44, 47-51, 54, 55, 68 and 69a-c or their esters 65a-h (scheme 3). Compound 68 was treated with BrCH₂COOCH₃ to generate the ester 65i. All esters 65a-i were hydrolyzed to the final products 16, 20 and 23-32 under alkaline conditions (scheme 4). Urea 21 was prepared from 4-aminobenzoic acid (66) and 4-tert-butylphenylisocyanate (67) with NEt₃ and subsequent hydrolysis with lithium hydroxide (scheme 5). The free carboxylic acid 18 served for the preparation of amides 37-39 using ammonium chloride, methylammonium chloride or dimethylammonium chloride and EDC/DMAP. Reduction of 18 with LiAlH₄ yielded ethyl alcohol derivative 33 which was further converted to aldehyde 34 with PCC (scheme 6). The inverted amides 40, 41 and 56, the inverted sulfonamides 46 and 57 as well as N-acyl sulfonamide 45 were generated from 42, 44, and 69a with EDC/DMAP for carboxylic acid activation. Tetrazole 43 was available from nitrile 36 by cycloaddition of NaN₃ under Cu₂O catalysis (scheme 7). Oxidation of methylmercaptane 51 to sulfoxide 52 and sulfone 53 was achieved using suitable equivalents of meta-chloroperbenzoic acid (mCPBA, scheme 8) and, finally, preparation of methoxy derivatives 76a-b according to the standard procedure (scheme 3) and their demethylation with BBr₃ yielded phenolic derivatives 77 and 78 (scheme 9).

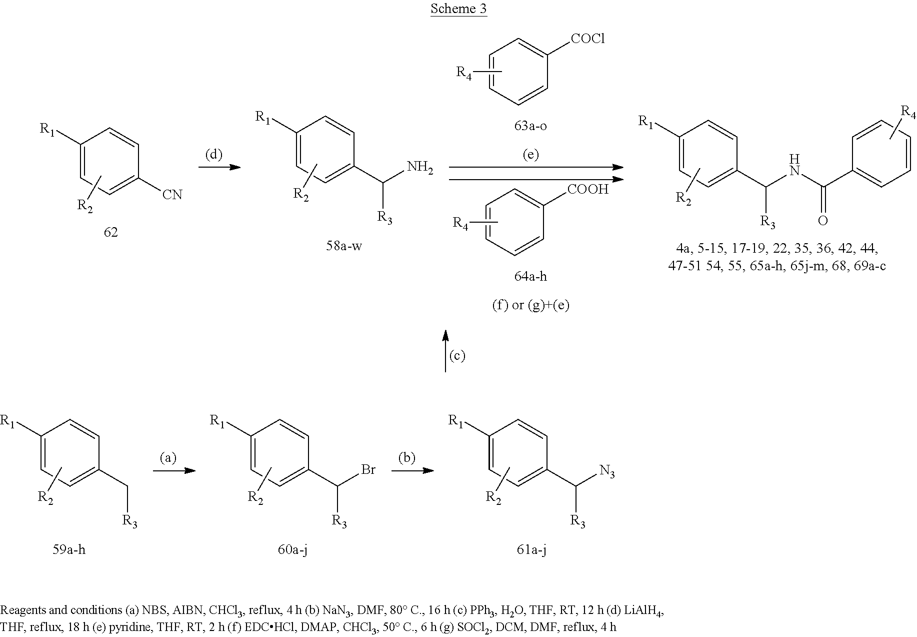

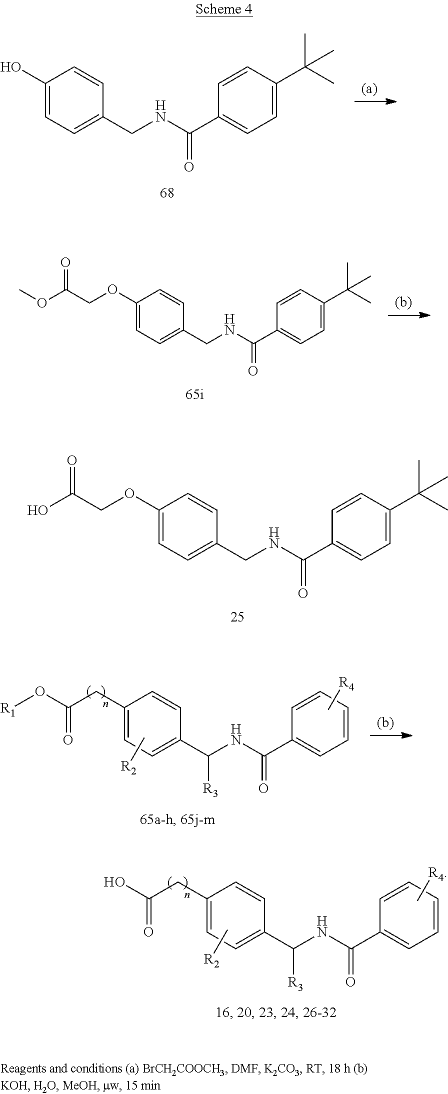

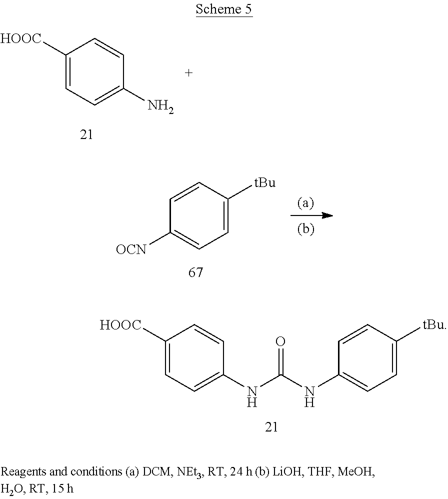

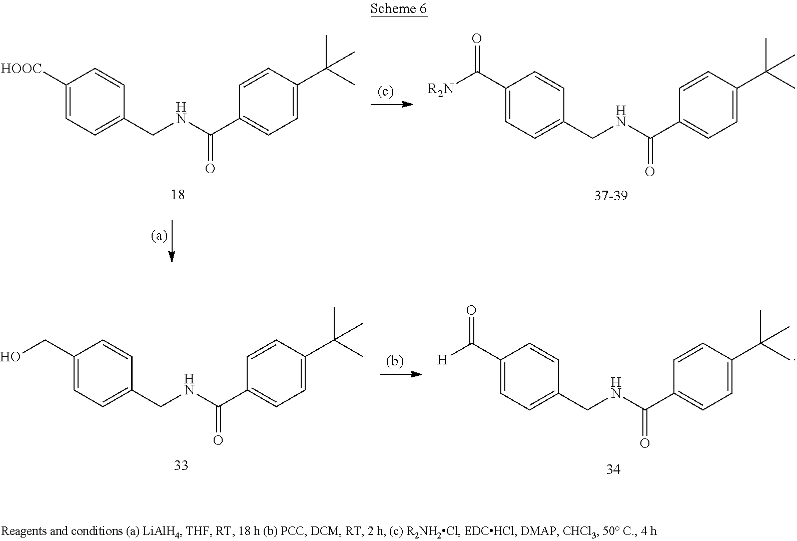

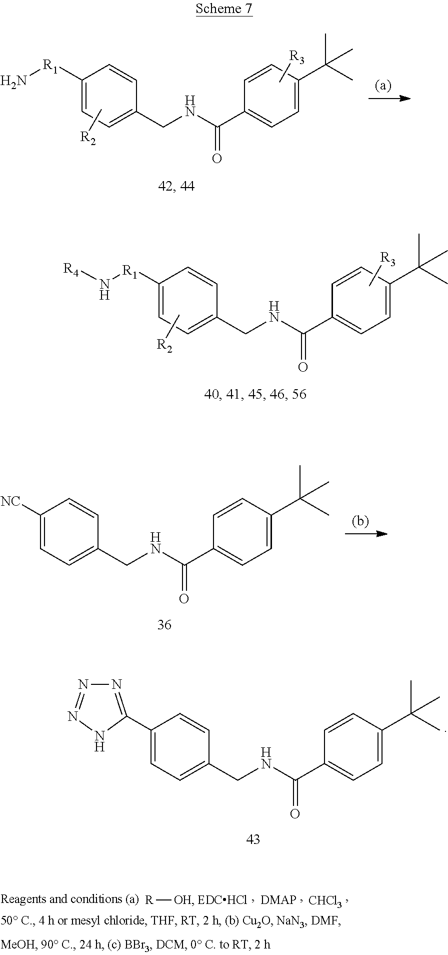

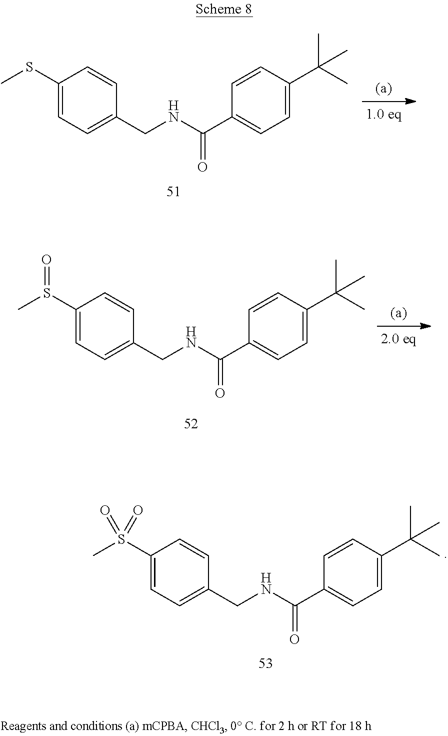

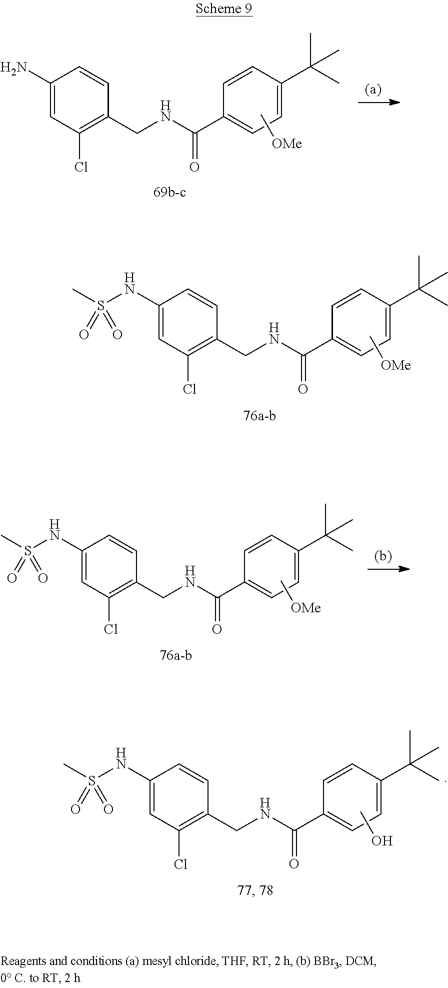

Compounds are similar as those disclosed in WO 2018/215610 A1 and in particular in tables 1 to 8 in the example section, which tables are incorporated herein by reference. The preferred compound of the invention is compound 57 therein and herein above in comparative example 1. Compound 57 is also denoted as DM509.

### Example 1: Bifunctional FXR Agonist/sEHi, DM509 Prevented Renal Fibrosis in UUO Model

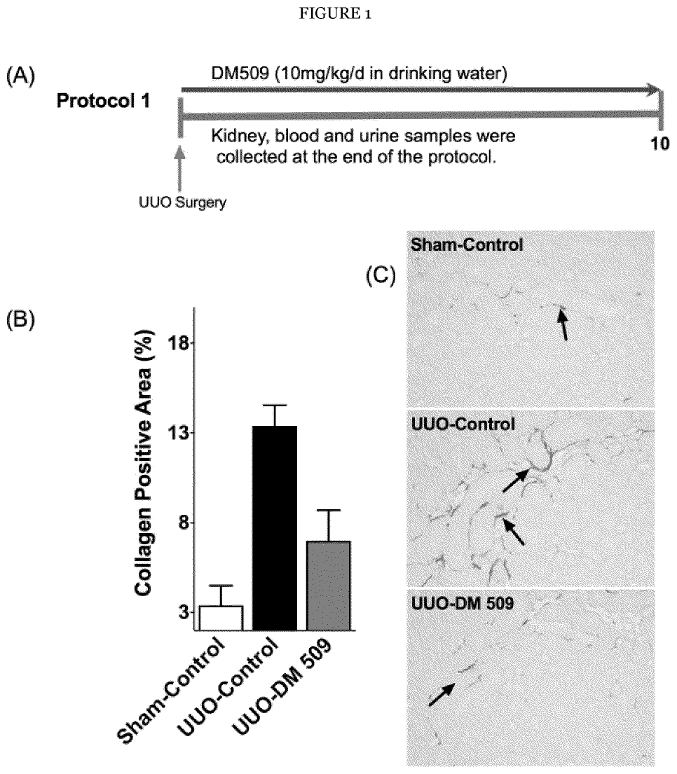

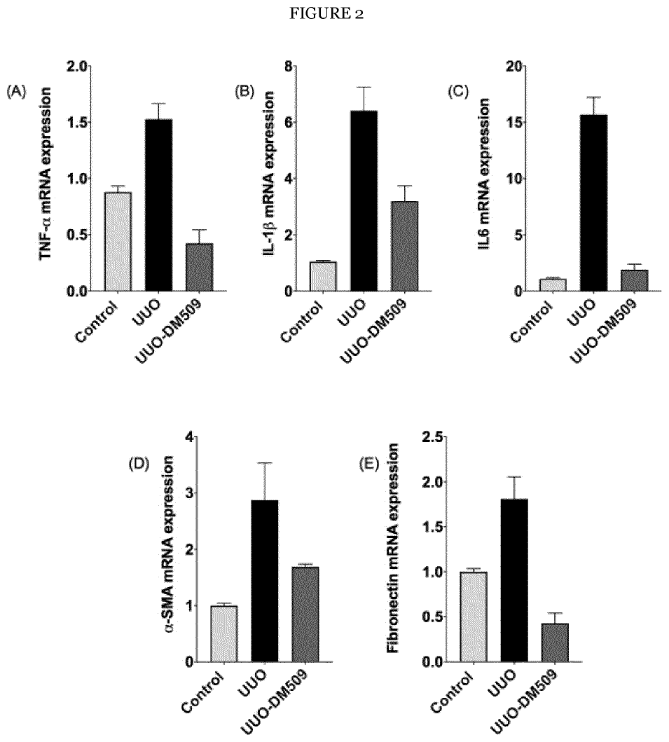

Mice with unilateral ureteral obstruction (UUO) developed marked renal fibrosis with 76% higher collagen positive renal area and 2-3-fold higher mRNA expression of fibrotic markers α-SMA and fibronectin compared to Sham. In UUO mice, DM509 pre-treatment prevented renal fibrosis in UUO with 54% lower collagen positive area and 40-60% lower renal mRNA expression of fibrotic markers than vehicle (FIG. 1B and FIG. 2D-E). The kidney of UUO mice also had 1.5 to 15-fold higher renal mRNA expression of inflammatory markers compared to Sham. DM509 pre-treatment resulted 50-80% lower expressions of these markers in UUO mice (FIG. 2A-C).

### Example 2: DM509 Mitigated Renal Fibrosis and Epithelial-to-Mesenchymal Transition (EMT) in UUO Model

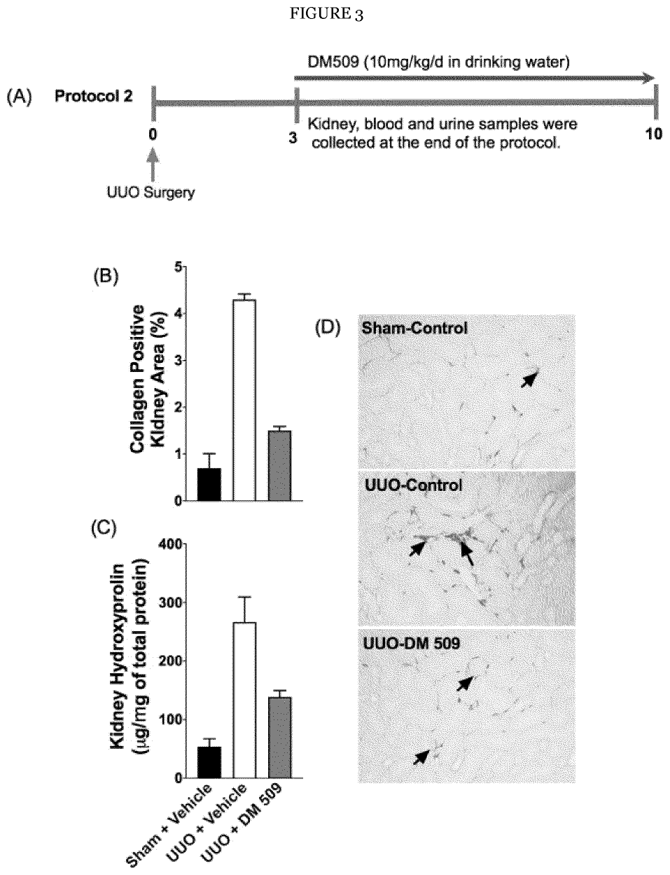

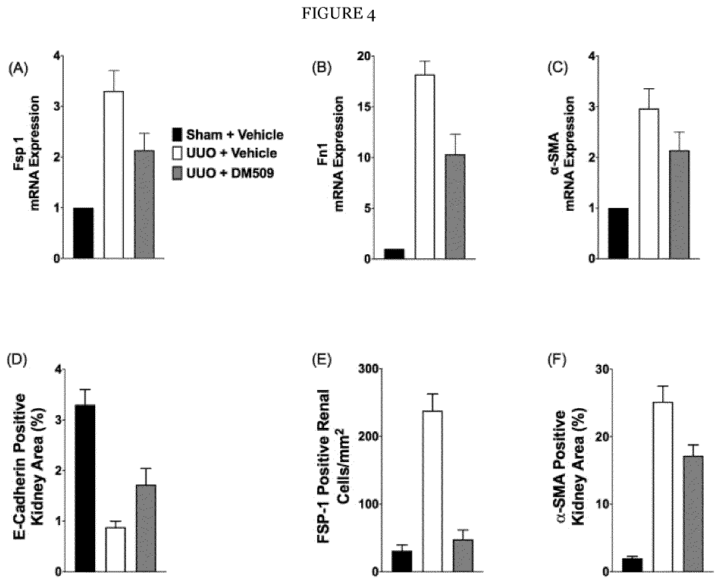

The UUO mice developed marked renal fibrosis after 3 days of UUO surgery with 75-85% higher collagen positive area and renal hydroxyproline content compared to Sham and DM509 markedly mitigated renal fibrosis in the UUO mice (FIG. 3B-D). DM509 mitigated EMT which is an important pathophysiological event of fibrogenesis. In UUO mice, markedly 60-90% higher mesenchymal (FSP-1, fibronectin and α-SMA) and a 70% lower epithelial (E-cadherin) marker expressions in the kidney indicated enhanced EMT. DM509 treatment markedly attenuated EMT by reducing mesenchymal and increasing epithelial marker expressions in the kidney of UUO mice (FIG. 4A-F).

### Example 3: DM509 Mitigated Renal Tubular Injury in UUO Renal Fibrosis Model

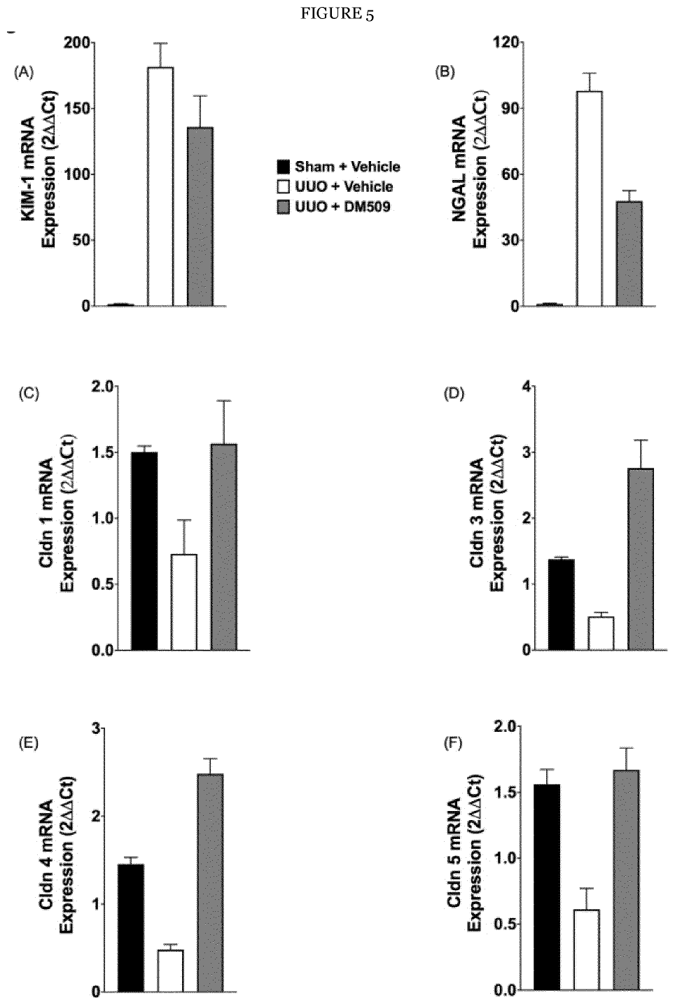

Renal tubular injury plays and important role in the pathophysiology of renal fibrosis and demonstrated marked beneficial effect of the novel bifunctional molecule DM509 in mitigating renal tubular injury in UUO mice. Renal mRNA expression of tubular injury markers kidney injury molecule-1 (KIM-1) and NGAL were 90-180 folds higher in vehicle treated UUO mice and DM509 treatment mitigated this marked elevation of KIM-1 and NGAL expressions in the kidney of UUO mice. The UUO mice demonstrated marked 40-60% decrease in the mRNA expression of claudins that are important for epithelial cellular integrity of renal tubule and their loss contributes to tubular injury. Interestingly, DM509 treatment re-stored the mRNA expressions of claudins 1,3,4 and 5 in the kidney of UUO mice (FIG. 5A-F).

### Example 4: DM509 Mitigated Renal Inflammation in UUO Renal Fibrosis Model

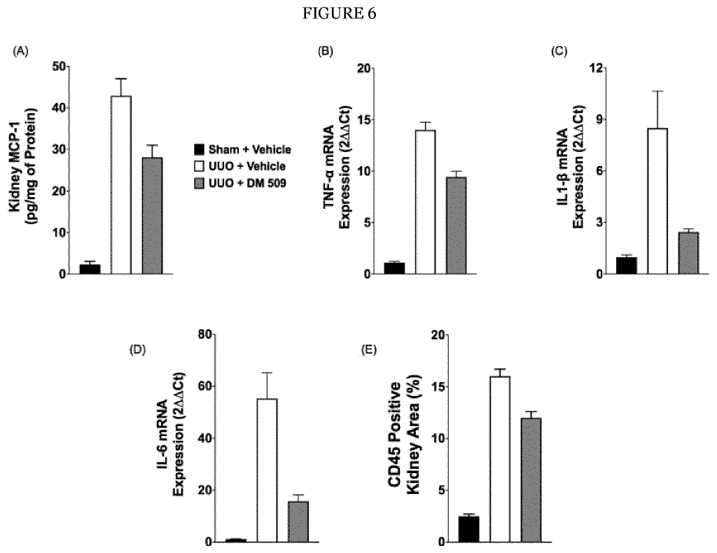

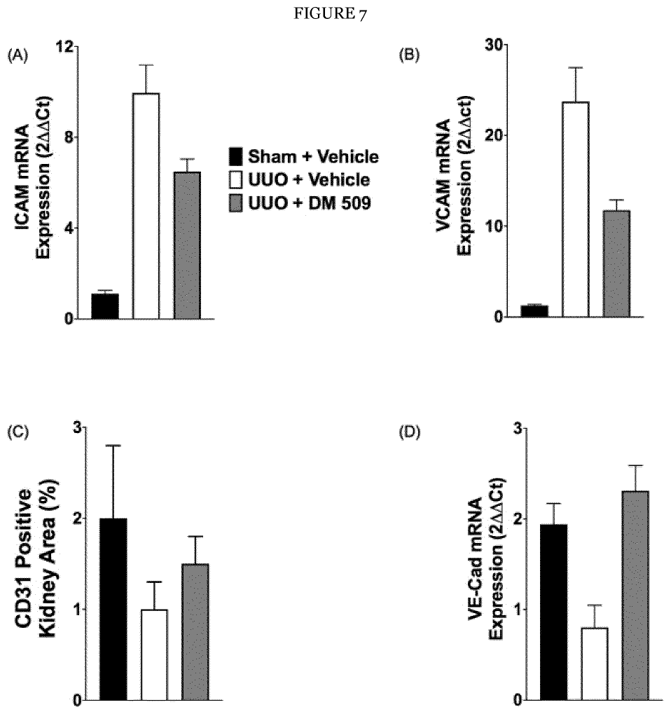

The UUO mice demonstrated marked renal inflammation, which is critically linked to renal fibrosis. Elevated kidney level of MCP-1 and 8-50-folds higher renal mRNA expression of prominent profibrotic cytokines (TNF-α, IL1β and IL6) is demonstrated in UUO mice. DM509 markedly mitigated renal inflammation by reducing renal MCP-1 level and cytokine expressions in the kidney of UUO mice. Renal inflammation in the UUO mice was also associated with markedly higher renal level of CD45 positive inflammatory cells (expressed as CD45 positive kidney area), and DM509 reduced infiltration of these cells in UUO mice kidney (FIG. 6A-E).

### Example 5: DM509 Mitigated Renal Vascular Damage in UUO Renal Fibrosis Model

Renal vascular injury is an important event that contribute to renal fibrosis and UUO mice demonstrated vascular injury. Renal mRNA expression of vascular inflammatory markers ICAM and VCAM were 10-25-fold higher in the UUO mice and DM509 mitigated elevated expression of these inflammatory markers in UUO kidney. The UUO mice also had markedly lower renal mRNA expression of VE-cadherin and decreased expression of CD31 positive cells in the UUO mice kidney. Such a lowered level of VE-cadherin and CD31 positive cells indicated vascular injury in the UUO mice. Interestingly, the novel bifunctional molecule DM509 markedly mitigated the decrease of VE-cadherin and CD31 positive cells and demonstrated its ability to reduce vascular injury during renal fibrosis (FIG. 7A-D).

### Example 6: Generation of an Derivative Class of the Herein Above Disclosed Dual Inhibitors of Soluble Epoxide Hydrolase (sEH) and Farnesoid X Receptor (FXR)

Similar to the above methods of compound synthesis, an alternative compound class replaces the aromatic ring in general formula (I) with another aryl or preferably heteroaryl. A preferred compound of the alternative class of dual inhibitors is the following compound.

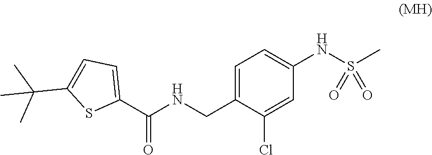

The following inhibitory activity of the most preferred compounds DM (DM509) and MH could be observed (see table 1).

TABLE 1
Inhibitory sEH and FXR Activity of Selected Compounds

| ID | structure | sEH | FXR |
|---|---|---|---|
| DM | 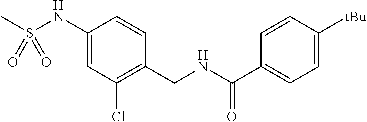 [SMILES: CC(C)(C)c1ccc(C(=O)NCc2ccc(NS(C)(=O)=O)cc2Cl)cc1] | IC₅₀ (enzyme) 0.004 μM IC₅₀ (cellular) 0.001 μM | partial agonist EC₅₀ 0.02 μM efficacy 35% of GW4064 K_{d} 0.13 μM |
| MH | 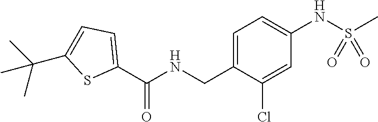 [SMILES: CC(C)(C)c1ccc(C(=O)NCc2ccc(NS(C)(=O)=O)cc2Cl)s1] | IC₅₀ (enzyme) < 0.001 μM IC₅₀ (cellular) < 0.001 μM | selective modulator K_{d} 1.4 μM |

### Example 7: In Vivo Effect of Selected Compounds DM and MH in a NASH Model

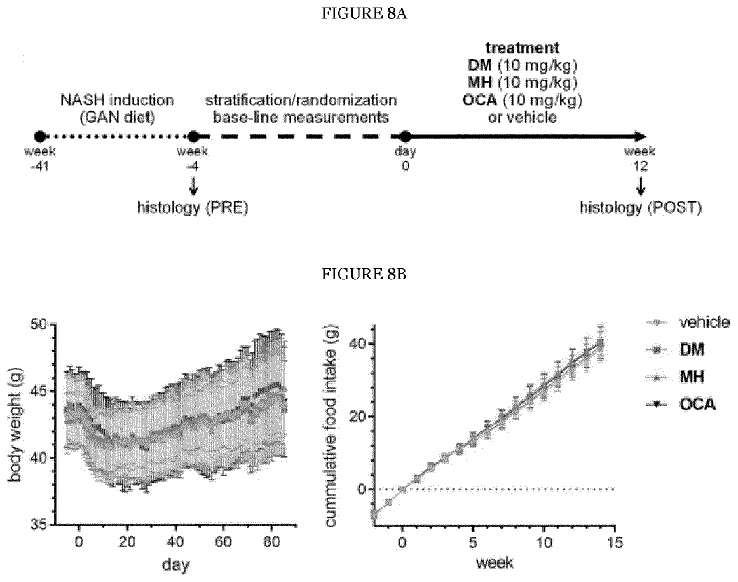

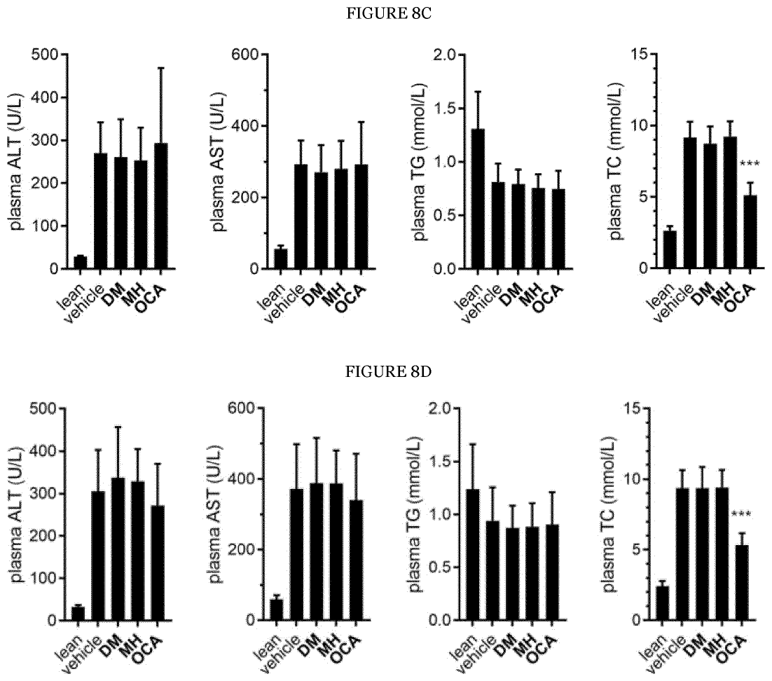

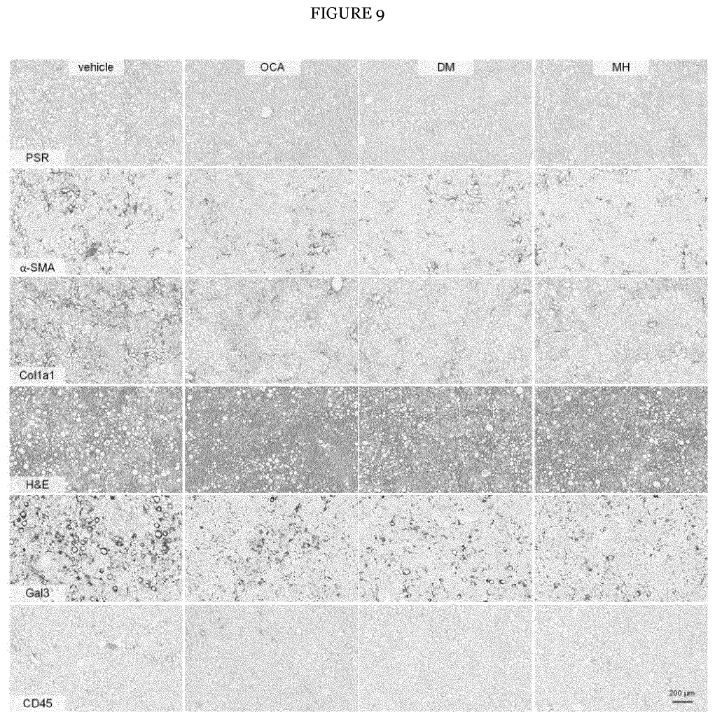

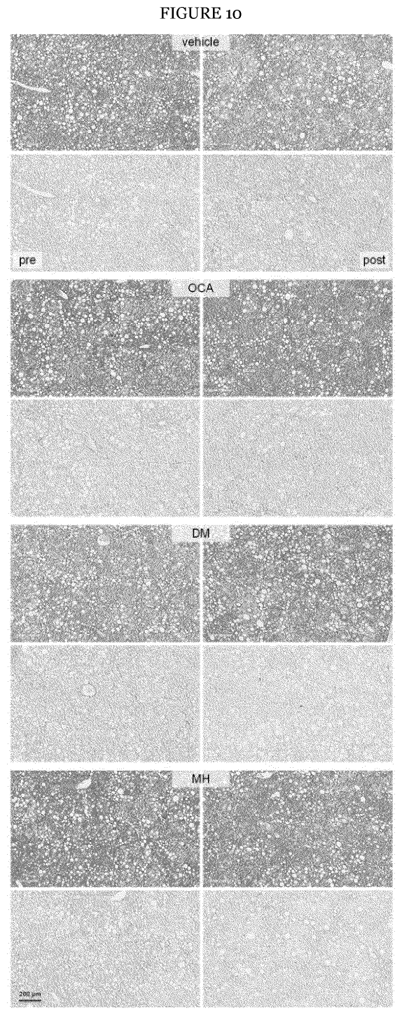

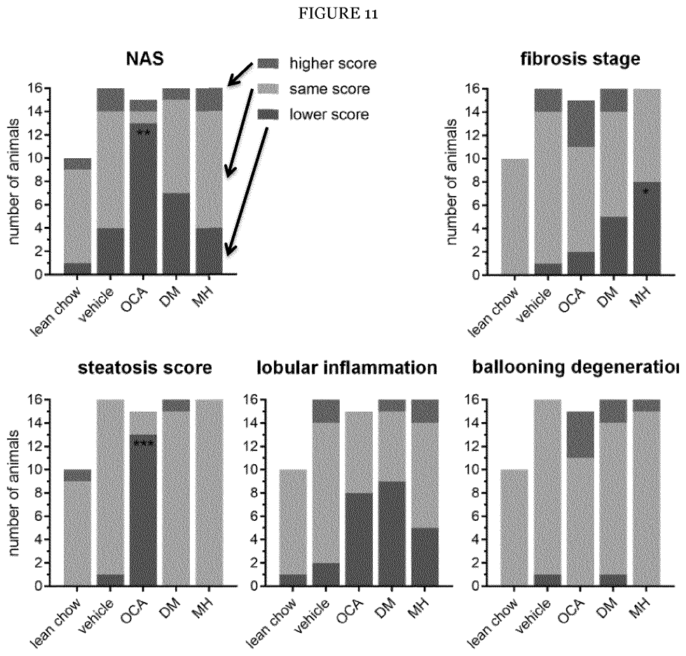

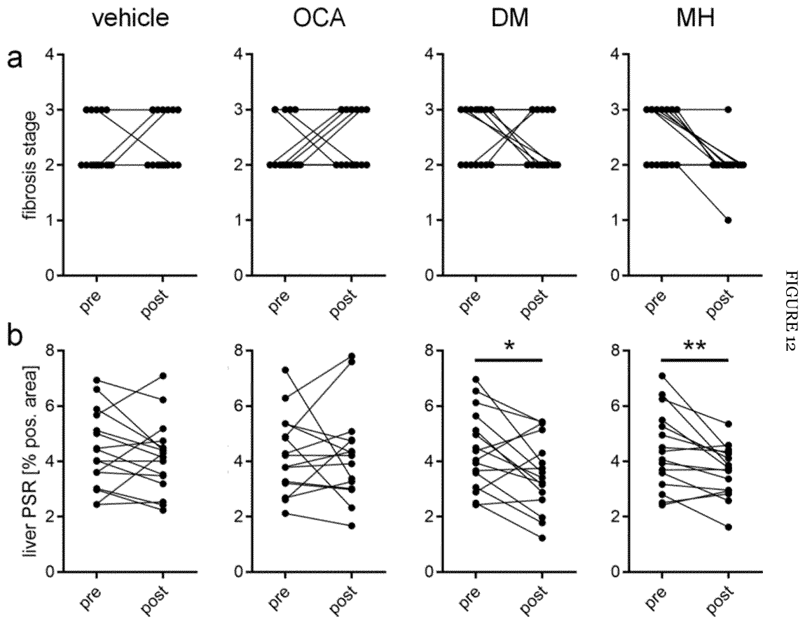

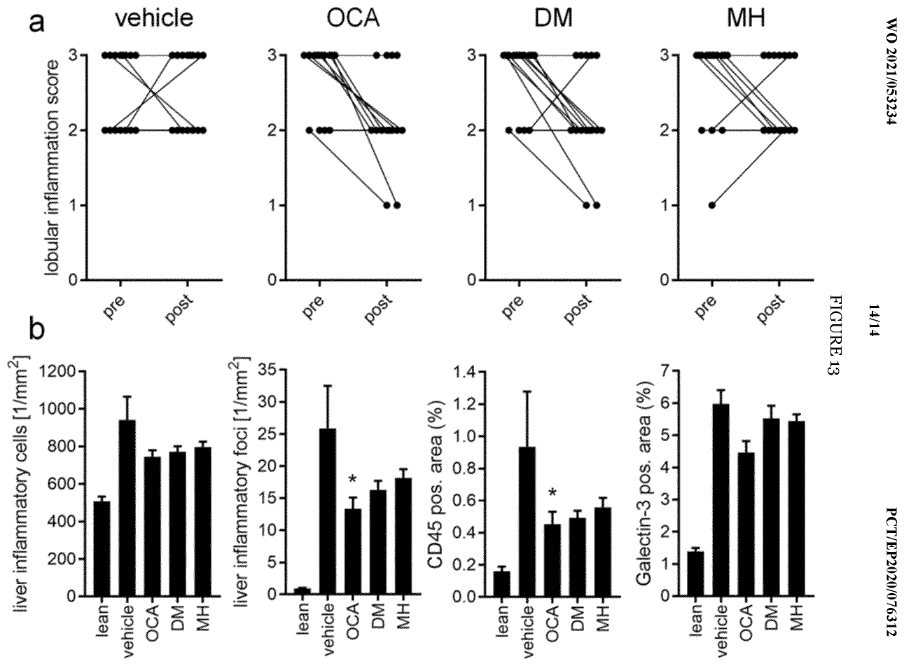

In vivo effects of the compounds (DM and MH) were examined in a NASH model which is outlined by the timeline in FIG. 8A. 41 weeks before treatment NASH was induced in C57BL/6JRj mice by using the Gubra Amylin NASH Diet. After stratification, randomization and base-line measurements the mice were divided into four groups (16 mice per group). Each group was either dosed with 10 mg/kg of DM or MH, 30 mg/kg Obeticholic Acid or vehicle for 12 weeks respectively. For the duration of the study there were neither differences in cummulative daily food intake nor in body weight among the treatment groups (FIG. 8B). After four weeks of treatment (FIG. 8C) and after termination (FIG. 8D) biochemical parameters were determined to examine and compare liver health (plasma alanine aminotransferase and plasma aspartate aminotransferase) and lipid homeostasis (plasma triglycerides and plasma total cholesterol) among treatment groups and healthy mice.

Furthermore, livers of all animals were analyzed by histopathology and the use of immuno-histochemistry (FIG. 9 and FIG. 10). FIG. 11 shows a comparison of post-treatment scores for each individual animal and their pre-treatment scores, comparing the progression or reversal of NAS, steatosis, fibrosis, lobular inflammation or ballooning degeneration respectively. A comparison of fibrosis stages can be found in FIG. 12, which displays the changes in fibrosis scores and changes in liver PSR positive area of each individual mouse from pre- to post-treatment within the different treatment groups. Inflammatory effects in the treatment groups are shown in FIG. 13a in a similar manner including a post-treatment histopathological analysis of inflammatory markers for the different treatment groups in FIG. 13b to investigate the anti-inflammatory in vivo activity of the compounds.

### SUMMARY

DM509 acts simultaneously as a farnesoid X receptor agonist and a soluble epoxide hydrolase inhibitor. C₅₇BL/6J male mice went through either UUO or sham surgery (n=8/group). Mice were treated with either DM509 (10 mg/kg/d) or vehicle given in drinking water. Preventive treatment demonstrated that DM509 decreased UUO kidney fibrosis. Next, interventional DM509 treatment was started three days after UUO induction and continued for 7 days. Plasma and kidney tissue was collected at the end of the experimental period. Several biochemical, histopathological, immunohistopathological, and gene expression studies were carried out to determine the antifibrotic actions for DM509. The UUO group exhibited elevated BUN compared to the sham group (77±10 vs 44±15 mg/dL). DM509 interventional treatment reduced BUN by 40%. UUO mice demonstrated marked renal fibrosis with higher kidney hydroxyproline content (267±46 vs. 53±14 μg/mg protein), collagen positive area (4.3±0.1% vs. 0.7±0.3%) compared to sham group. DM509 reduced hydroxyproline content by 41% and collagen positive area by 65%. Renal inflammation was evident in UUO mice with elevated kidney MCP-1, renal CD45 immune cell positive infiltration, and renal profibrotic gene expression (TNF-α, IL-6, IL-1β). Interventional DM509 treatment reduced renal inflammation in UUO mice. Renal fibrosis in UUO was associated with epithelial-to-mesenchymal transition (EMT) as UUO mice exhibited a 2 to 20-fold higher protein and gene expression of mesenchymal markers α-SMA, FSP-1, and FN, as well as a marked decrease in the epithelial marker, E-cadherin compared to sham group. UUO mice with interventional DM509 treatment had markedly reduced EMT. UUO mice also had tubular epithelial barrier injury with increased renal KIM-1, NGAL expression and lower claudin-1 and -5 expression compared to sham. DM509 interventional treatment reduced tubular injury markers by 25-50% and maintained tubular epithelial integrity by restoring claudin expressions in UUO mice. Vascular inflammation was evident in UUO mice with 9 to 20-fold higher renal ICAM and VCAM gene expression compared to sham that was reduced by 40-50% with DM509 interventional treatment. Peritubular vascular density assessed by CD31 expression was reduced by 35% in UUO mice and DM509 mitigated vascular density loss. In conclusion, these data reveal that DM509 is a dual acting antifibrotic drug that combats epithelial and vascular renal fibrotic pathological events.

### Materials and Methods

### Animal Experiments

This study was approved and conducted according to guidelines of the Institutional Animal Care and Use Committee, Medical College of Wisconsin. The Biomedical Resource Center at the Medical College of Wisconsin housed animals with free access to water and food and a 12/12 h light-dark cycle. Male C57B1/6J mice (8-10 weeks old) were purchased from Jackson Laboratories, Bar Harbor, Me. Mice were administered 2.0% isoflurane to induce anesthesia prior to UUO surgery. UUO surgery was conducted with by obstructing the left ureter proximal to the renal pelvis using a 6-o silk tie (Sharma et al., 2016; Kim et al., 2015). Mice with sham surgery went through the same procedure as the UUO mice except that the ureter was not ligated. Two experimental protocols were used to determine renal antifibrotic actions of DM509 and in both protocols the mice were subjected to Sham surgery (n=6/protocol) or Unilateral Ureteral Obstruction (UUO) surgery to induce renal fibrosis. In Protocol 1, mice went through UUO surgery after 24 hours pre-treatment of DM509 (10 mg/kg/d, p.o., n=8) or vehicle (n=8) and blood and kidney tissue were collected at day 10 of the UUO surgery. Mice of the Protocol 2 treated with DM509 (n=8) or vehicle (n=8) after 3 days of UUO surgery and continued for ₇ days followed by blood and kidney tissue collection. Experimental protocols are shown in FIGS. 1(A) and 3(A). Kidney samples for histological and immune-histological studies were fixed in 10% buffered formalin and stored at room temperature. Serum and tissue samples for biochemical and gene expression analysis were snap-frozen in liquid nitrogen and stored at −800 C.

### Biochemical Analysis

Kidney hydroxyproline levels were measured using a commercially available kit (Sigma Aldrich, USA). Blood urea nitrogen (BUN) is measured using a kit from Arbor Assay, USA and monocyte chemoattractant protein-1 (MCP-1) was measured using kit from R&D Systems, Inc., USA.

### Real-Time PCR Analysis

Renal mRNA expression for several fibrotic [fibronectin, α-smooth muscle actin (α-SMA), fibroblast specific protein-1 (FSP-1], tubular injury [NGAL, kidney injury molecule-1 (KIM-1), Claudins1,3,4,5], inflammatory (TNF-α, IL1β, IL-6, ICAM, VCAM) were studied by Real Time-PCR (RT-PCR) analysis. RNeasy Mini Kit (QIAGEN, Calif., USA) was used according to the manufacturer's protocol and messenger RNA (mRNA) was prepared from each sample homogenate. The mRNA samples were quantified spectrophotometrically and 1 μg of total RNA was reverse-transcribed to cDNA using iScript™ Select cDNA Synthesis Kit (Bio-Rad, Hercules, Calif., USA). Gene expression was quantified by iScript One-Step RT-PCR Kit with SYBR green using the MyiQ™ Single Color Real-Time PCR Detection System (Bio-Rad Laboratories, Hercules, Calif., USA). Dissociation curve analysis was accomplished with iQ5 Optical System Software, Version 2.1 (Bio-Rad Laboratories, Hercules, Calif., USA), and each amplified sample analysed for homogeneity. Denaturation was done at 95° C. for 2 min followed by 40 cycles conducted at 95° C. for 10 s and at 60° C. for 30 s. All samples were run in triplicate and fold change in gene expression compared to controls determined by comparative threshold cycle (Ct) method. Target gene expression levels were determined by normalizing Ct values to two housekeeping genes. Statistical analyses were carried out using six samples from each experimental group and comparing to the control group.

### Histopathology

Renal tissues were fixed in 10% formalin, sectioned a 5 μm thickness, mounted on slides, and stained with Periodic Acid-Schiff (PAS) (Acros Organics, Fairlawn, N.J.) or Picrosirius Red (PSR) (Alfa Aesar, Tewksbury, Mass.) for histological examination at a 200× magnification using NIS Elements AR version 3.0 imaging software (Nikon instruments Inc., Melville, N.Y., USA). PAS-stained tissue was evaluated for tubular injury whilst PSR was used to determine collagen-positive renal interstitial fibrosis levels. Histopathological changes were scored as published previously and scores presented as a percentage area-fraction relative to the total area analysed. Tubular injury (PAS-cast areas) and interstitial fibrosis (PSR-collagen positive areas) scoring was performed in a blinded fashion by two observers.

### Immunohistopathological Analysis

Kidney histological slides were deparaffinized and re-hydrated followed by overnight incubation with anti-α-SMA antibody (moo, Santa Cruz Biotechnology, USA), anti-FSP-1 (1:100, Cell Signaling Technology, USA), anti-E-cadherin, -CD31, -CD45 were purchased from Abcam, USA and were used at dilutions between 1:50-1:200. On the second day, the slides were washed and incubated with biotinylated secondary antibody (1:150-1:200) for 1 hour. Immunopositivity was determined from avidin-biotinylated HRP complex (Vectastain ABC Elite kit, Vector Laboratories, USA) followed by counterstaining with hematoxylin. Stained histological sections were visualized at 200-400× magnification with a light microscope and analyzed using Nikon NIS Elements Software (Nikon Instruments Inc., Melville, N.Y., USA). Kidney immune-positive areas specific for each target protein used were expressed as the percentage area relative to total area analysed and this was carried out by two observers in blinded fashion.

### Statistical Analysis

All data are expressed as mean ±S.E.M. GraphPad Prism® Version 4.0 software was utilized to conduct a one-way ANOVA followed by Tukey's post-hoc test to establish statistical significance between groups (GraphPad Software Inc, La Jolla, Calif., USA). Two-tailed unpaired Student's t-test was applied to determine statistical significance between groups. A P<0.05 was deemed significant.

### REFERENCES

The references are:
1) Chiang C W, Lee H T, Tarng D C. et al. (2015). Kuo K L, Cheng L C, Lee T S. Genetic deletion of soluble epoxide hydrolase attenuates inflammation and fibrosis in experimental obstructive nephropathy. Mediators Inflamm. 2015, 693260.
2) Collins, A J, Foley, R N, Gilbertson, D T. et al. (2009). The state of chronic kidney disease, ESRD, and morbidity and mortality in the first year of dialysis. Clin. J. Am. Soc. Nephrol. 4 (Suppl1), S5-S11.
3) De Zeeuw, D, Akizawa, T, Audhya, P. et al. (2013). Bardoxolone methyl in type 2 diabetes and stage 4 chronic kidney disease. N. Engl. J. Med. 369, 2492-2503
4) Fried, L F, Emanuele, N, Zhang, J H. et al. (2013). Combined angiotensin inhibition for the treatment of diabetic nephropathy. N. Engl. J. Med. 369, 1892-1903.
5) Gai Z, Chu L, Xu Z. et al. (2017). Farnesoid X receptor activation protects the kidney from ischemia-reperfusion damage. Sci Rep. 7, 9815.
6) Imig, J D. Epoxides and soluble epoxide hydrolase in cardiovascular physiology. Physiol Rev. 92, 101-30.
7) Jiang, T, Wang, X X, Scherzer, P. et al. (2007). Farnesoid X receptor modulates renal lipid metabolism, fibrosis, and diabetic nephropathy. Diabetes 56, 2485-2493.
8) Kim J, Imig J D, Yang J et al. (2014). Inhibition of soluble epoxide hydrolase prevents renal interstitial fibrosis and inflammation. Am J Physiol Renal Physiol. 307, F971-80.
9) Kim J, Yoon S P, Toews M L. et al. (2015). Pharmacological inhibition of soluble epoxide hydrolase prevents renal interstitial fibrogenesis in obstructive nephropathy. Am J Physiol Renal Physiol. 308, F131-9.
10) Li S, Ghoshal S, Sojoodi M. et al. (2019). The farnesoid X receptor agonist EDP-305 reduces interstitial renal fibrosis in a mouse model of unilateral ureteral obstruction. FASEB J. 33, 7103-7112.
11) Liu, Y. (2006). Renal fibrosis: new insights into the pathogenesis and therapeutics. Kidney Int. 69, 213-217.
12) Mann, J F, Green, D, Jamerson, K. et al. (2010). Avosentan for overt diabetic nephropathy. J. Am. Soc. Nephrol. 21, 527-535.
13) Suh J M, Yu CT, Tang K, et al. (2006). The expression profiles of nuclear receptors in the developing and adult kidney. Mol Endocrinol 20, 3412-3420.
14) Verbeke L, Mannaerts I, Schierwagen R. et al. (2016). FXR agonist obeticholic acid reduces hepatic inflammation and fibrosis in a rat model of toxic cirrhosis. Sci Rep. 6, 33453.
15) Watanabe M, Houten S M, Wang L, et al. (2004). Bile acids lower triglyceride levels via a pathway involving FXR, SHP, and SREBP-1C. J Clin Invest. 113, 1408-1418.
16) Zhao, K, He, J, Zhang Y. et al. (2016). Activation of FXR protects against renal fibrosis via suppressing Smad3 expression. Sci. Rep. 6, 37234.
17) Zhong, J, Yang, H C, Fogo, A B. (2017). A perspective on chronic kidney disease progression. Am. J. Physiol. Renal Physiol. 312, F375-F384.

1-26. (canceled)

27. A method for the treatment or prevention of a kidney disease and/or a fibrotic disease in a subject, the method comprising a step of administering a compound having the formula I: 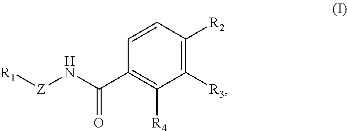
wherein R₁, R₂, R₃ and R₄ are independently selected from H, an unsubstituted, monosubstituted, or polysubstituted C₁-C₁₈ alkyl or heteroalkyl, wherein said alkyl is straight, branched or cyclic, a unsubstituted, monosubstituted or polysubstituted C₁-C₁₈ alkenyl or heteroalkenyl, wherein said alkenyl is straight, branched or cyclic, an unsubstituted, monosubstituted, or polysubstituted aryl or heteroaryl, an unsubstituted, monosubstituted, or polysubstituted benzyl group, an acyl group, a sugar or another acetal, and a sulfonyl group, and/or R₂, R₃ and/or R₄ form together a nonsubstituted, monosubstituted, or polysubstituted ring;
Z is C with or without any substitution;
or an isomer, prodrug, or derivative thereof, or a pharmaceutically acceptable salt or solvate of these compounds.

28. The method according to claim 27, wherein R₂ is C₁-C₁₀ alkyl, R₃ is H, —OH or —OMe, and R₄ is H, —OH or —OMe.

29. The method according to claim 27, wherein R₁ is a mono or polysubstituted aryl.

30. The method according to claim 29, wherein R₁ is selected from:

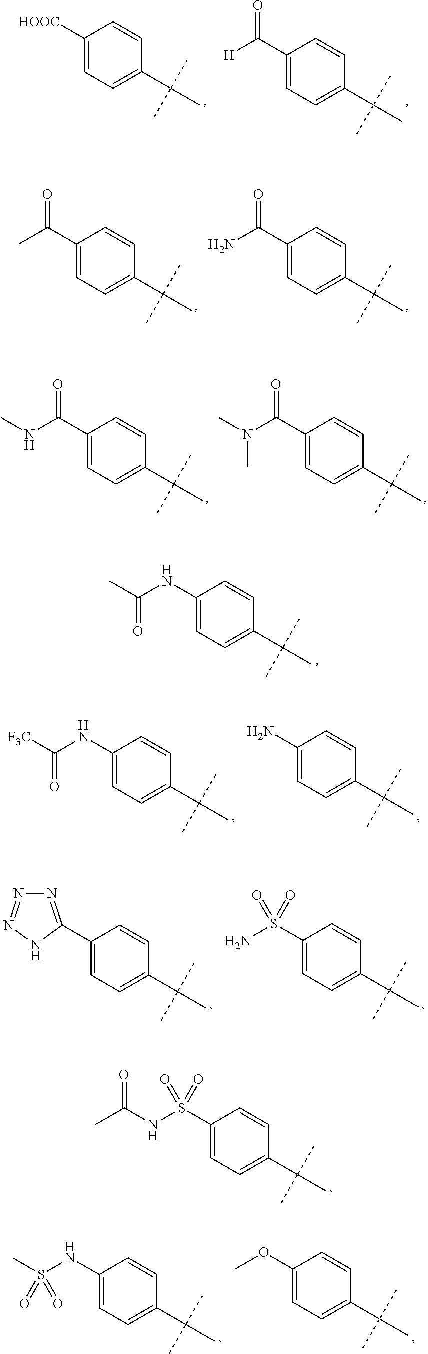

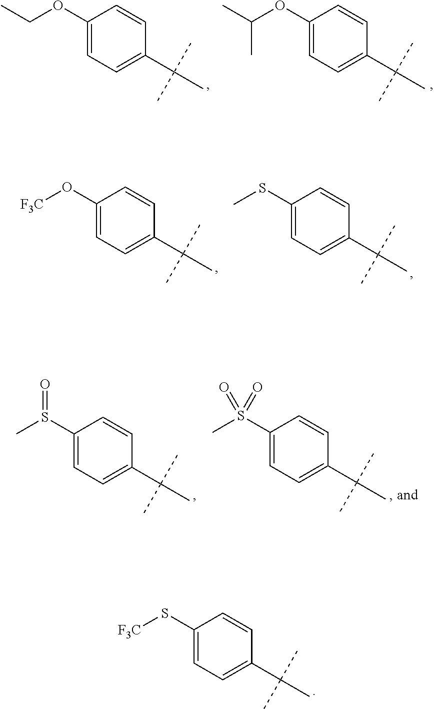

31. The method according to claim 27, wherein R₁ is selected from the group consisting of: 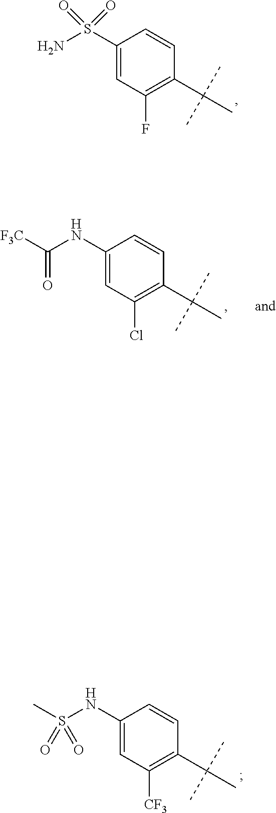
and
wherein Z is C, R₂ is —C(CH₃)₃, and R₃ is H.

32. The method according to claim 27, wherein R₁ is selected from the group consisting of: 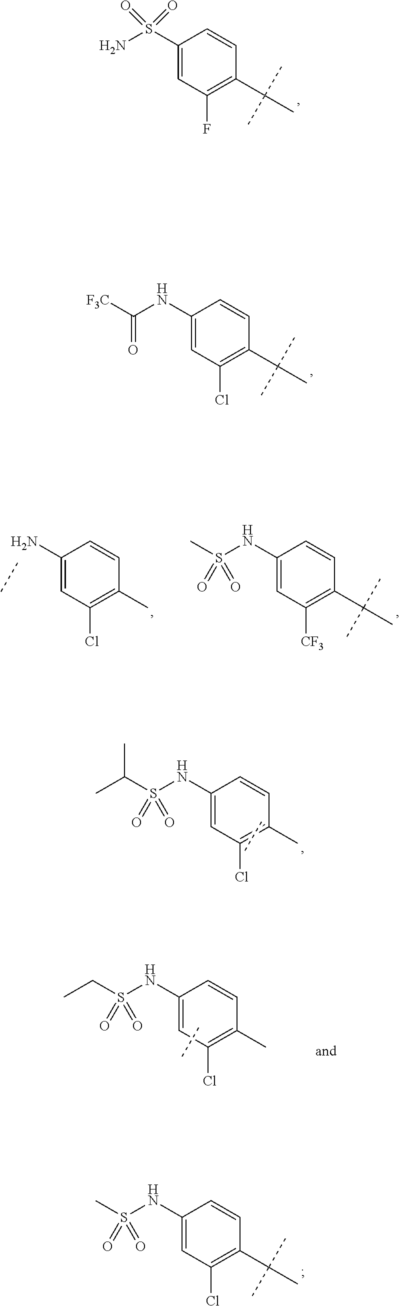
and
wherein Z is C, R₃ is H or OH, and R₄ is H or OH, but R₃ and R₄ are not both OH; and
wherein R₂ is —C(CH₃)₃ or —N(CH₃)₂, or R₂ is:

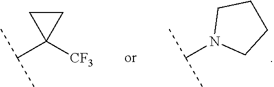

33. The method according to claim 27, wherein the compound is a farnesoid X receptor (FXR) agonist and a soluble epoxide hydrolase (sEH) inhibitor.

34. The method according to claim 27, wherein the compound has the formula: 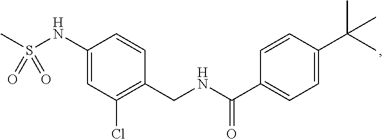 [SMILES: CC(C)(C)c1ccc(C(=O)NCc2ccc(NS(C)(=O)=O)cc2Cl)cc1]
or an isomer, prodrug, or derivative thereof, or a pharmaceutically acceptable salt or solvate of this compound.

35. The method according to claim 27, wherein the kidney disease is a chronic kidney disease (CKD).

36. The method according to claim 27, wherein the treatment comprises the administration of a therapeutically effective amount of the compound to the subject suffering from, or suspected to suffer from, the kidney disease.

37. The method according to claim 27, wherein the kidney disease is a progression of CKD.

38. The method according to claim 27, wherein the treatment is a prevention of progression of CKD.

39. The method according to claim 27, wherein the kidney disease is, or involves in its pathology, renal fibrosis, renal inflammation, and/or renal tubular injury.

40. The method according to claim 27, wherein the compound reduces or mitigates/reduces renal epithelial to mesenchymal transition.

41. A pharmaceutical composition comprising a compound recited in claim 27, together with a pharmaceutical acceptable carrier and/or excipient.

42. The pharmaceutical composition according to claim 41, wherein the compound is present in an amount which is, when the pharmaceutical composition is administered to a subject, therapeutically effective to treat a kidney disease.

43. A method for the treatment or prevention of a kidney disease and/or fibrotic disease, wherein the method comprises a step of administering to the subject a therapeutically effective amount of a pharmaceutical composition, the pharmaceutical composition comprising a compound recited in claim 27 together with a pharmaceutical acceptable carrier and/or excipient.

44. The method according to claim 27, wherein the compound reduces or mitigates/reduces renal epithelial to mesenchymal transition in the subject.

45. The method according to claim 27, wherein the subject is a human patient.

46. The method according to claim 27, wherein the fibrotic disease is Peyronie's disease, Raynaud's syndrome, psoriasis plaques, eczema, keloid scars, pulmonary fibrosis, liver fibrosis, renal fibrosis, or vascular fibrosis.
